SEQUENCE LISTING (8 sequences)

SEQ ID NO: 1 - protein, 20 aa, unidentified
CCCTCGTTTTGCCATGAACC

SEQ ID NO: 2 - protein, 20 aa, unidentified
CCAGTGTTCCAAGGTCCACA

SEQ ID NO: 3 - protein, 22 aa, unidentified
ACATTGCTGCACAAGGTCCCAG

SEQ ID NO: 4 - protein, 22 aa, unidentified
AACCATCAGGCATCTCTGTCGC

SEQ ID NO: 5 - protein, 19 aa, unidentified
ATTCCTATGTGGGCGACGA

SEQ ID NO: 6 - protein, 19 aa, unidentified
AATGGTGATGACCTGGCCG

SEQ ID NO: 7 - protein, 24 aa, synthetic construct
TTTTTTTTTTTTTTTTTTTTTTVN

SEQ ID NO: 8 - protein, 33 aa, unidentified
AAGCAGTGGTATCAACGCAGAGTACATRGRGRG

(19) United States
(12) Patent Application Publication
(10) Pub. No.: US 2024/0318354 A1
(43) Pub. Date: Sep. 26, 2024

(54) BARCODING NUCLEIC ACID MOLECULES DERIVED FROM INDIVIDUAL CELLS

(71) Applicant: VILNIUS UNIVERSITY, Vilnius (LT)

(72) Inventors: Linas MAZUTIS, Vilnius (LT); Greta LEONAVICIENE, Vilnius (LT); Denis BARONAS, Kaunas (LT)

(21) Appl. No.: 18/715,381

(22) PCT Filed: Dec. 1, 2022
(86) PCT No.: PCT/EP2022/084066

§ 371 Date: May 31, 2024

Related U.S. Application Data
(60) Provisional application No. 63/284,665, filed on Dec. 1, 2021.

(51) Int. Cl. C40B 70/00; C12Q 1/6806; C40B 50/00

(52) U.S. Cl. CPC C40B 70/00; C12Q 1/6806; C40B 50/00

(57) ABSTRACT

The present invention concerns a method for sequentially attaching a plurality of oligonucleotides to nucleic acids to produce barcoded nucleic acids, wherein the nucleic acids are obtained from a plurality of cells, wherein the plurality of cells are in a plurality of microcapsules, each microcapsule comprising a semi-permeable shell and a core.

### TECHNICAL FIELD

The present invention provides methods for single-cell analysis involving the compartmentalisation of single cells into microcapsules, the microcapsules having a semi-permeable shell and a core, and the performance of multistep reactions on the nucleic acids obtained from the cell within the microcapsule to produce barcoded nucleic acids. The barcoded nucleic acids can then be released from the microcapsule for further analysis/processing, such as library preparation and sequencing. The invention can be applied to the investigation (individually or simultaneously) of the transcriptome, epigenome, methylome and genome of single cells and can be performed in a high-throughput manner.

### BACKGROUND OF THE INVENTION

Over the last few years technological advances has enabled single-cell analytics of transcriptome [1-3], epigenome [4-9], methylome [10, 11], genome [12, 13], and targeted proteome [14, 15]. However, the integration of individual modalities into a unified single-cell multi-omics platform has proven to be a challenging task [16]. Although latest developments have achieved dual-omics (joined-profiling) [14, 15, 17-19], the ultimate goal in single-biology field remains to address the genomic, epigenomic and transcriptomic layers of information of individual cells at once and to do so in a high-throughput and affordable manner. Simultaneous multi-omics studies on millions of single-cells en masse are especially challenging not only due to technical difficulties associated with biological sample processing and efficient single-cell isolation, but also due to complex, multi-step and sequential biochemical reactions that need to be conducted on miniscule amounts of biomaterial. Most, if not all, state-of-the-art multi-omics techniques reported to-date are relying on three strategies that have conceptual and technical drawbacks:
1) in ultra-high-throughput methods nuclei are extracted from live cells, fixed with a cross-linking agent and processed via combinatorial barcoding e.g. split-pool. The “split-and-pool” method, was originally suggested in combinatorial chemical synthesis [20, 21], and different methods based on split-pool have been applied to profile mouse brain [19, 22], mammalian organogenesis [8, 23], and other biological systems [6, 24, 25]. However, the transcriptome-encoded information content that can be extracted from these methods is very sparse [23]. Therefore, the genetic information content that can be extracted from these methods is largely restricted to long, unprocessed RNAs [23]. Furthermore, in these methods the input material is fixed nuclei or, in some cases, fixed cells, (that have typically been fixed using the cross-linking agent paraformaldehyde). The use of cross-linking agents, unfortunately, degrades RNA molecules [26] and reduces the sensitivity by a few fold as compared to non-fixed samples [22, 27]. Another serious bottleneck of existing split-and-pool methods is uncontrolled sample loss; 50 to 90% of cells (nuclei) is lost “in process” while performing split-pool barcoding [18, 28]. As such, the existing split-pool methods have serious limitations that prohibits their broader use.
2) high-throughput methods based on microfluidic approaches [1-3, 22, 29] provide a higher sensitivity, since intact cells are isolated in droplets (or nano-wells), but at a cost of permitting only one type of reaction (e.g. RT or PCR). Once the cells are isolated, removing or replacing the reagents inside the compartments becomes a daunting task. Therefore, single-cell genomic assays in droplets are solely built on biochemical reaction conditions that are compatible with RT or PCR, and that do not compromise droplet stability [7, 14, 15].

Performing multi-step or higher order biochemical reactions is challenging due to incompatible requirements of different enzymes, inhibitory salt effects, inability to replace buffers, and an inability to remove unused reagents or add new ones.
3) in mid- and low-throughput methods such as using 96-well plates, the different types of biomolecules extracted from individual cells are physically separated from each other by biochemical tags, centrifugation, and other chemical or physical means [30-35]. While this provides a flexibility in designing the enzymatic assays, the relatively large reaction volumes reduce the capture rate of biomolecules. Moreover, large reaction volume directly translates into a high-cost of reagents per cell (>100-times higher as compared to high-throughput droplet methods). Therefore, single-cell omic assays in 96-well plate assays will find their use only for a limited number of applications such as deep sequencing of FACS-enriched cells [36], but it will remain prohibitively costly for multimodal-omics profiling of thousands and millions of single-cells.

Accordingly, there remains a need to provide improvements to the methods previously described in the art for the analysis of nucleic acids from single cells.

### SUMMARY OF THE INVENTION

The present invention provides a method for sequentially attaching a plurality of oligonucleotides to nucleic acids to produce barcoded nucleic acids, wherein the nucleic acids are obtained from a plurality of cells, wherein the plurality of cells are in a plurality of microcapsules, each microcapsule comprising a semi-permeable shell and a core, wherein each cell is in a separate microcapsule, the method comprising:
(a) lysing the cells within the microcapsules to release DNA and RNA inside the microcapsules;
(b) optionally in the microcapsule:
   (i) converting released cellular RNA to cDNA by reverse transcription; and/or
   (ii) fragmenting the released cellular DNA to produce fragmented DNA; and/or
   (iii) amplifying fragmented DNA and/or cDNA,
(c) applying a combinatorial indexing strategy to attach the plurality of oligonucleotides one at a time to the released cellular DNA, the released cellular RNA, the cDNA, and/or the fragmented DNA and/or amplified DNA and/or cDNA in each microcapsule to produce barcoded nucleic acids;
wherein the barcoded nucleic acids produced in each microcapsule comprise a sequence of oligonucleotides that is specific to that microcapsule.

In particular, the above method may be a method in which the combinatorial indexing strategy of (c) comprises:
(i) randomly distributing the plurality of microcapsules into a plurality of compartments, wherein each compartment comprises more than one microcapsule;
(ii) processing the microcapsules in each compartment to attach an oligonucleotide to the nucleic acid in the microcapsules, wherein a different oligonucleotide is utilised in each compartment;
(iii) pooling and randomly re-distributing the plurality of microcapsules into a further plurality of compartments, wherein each further compartment comprises more than one microcapsule, and processing the microcapsules in each compartment to attach a further oligonucleotide to the nucleic acid in the microcapsules, wherein a different oligonucleotide is utilised in each of the further compartments; and optionally repeating step (iii) one or more times.

Further embodiments of the invention are set out in the dependent claims.

The method of the invention is advantageous over the single-cell barcoding strategies of the prior art since it does not require the fixing of cells or nuclei and therefore the method can avoid the loss of material that is associated with this processing step. In particular, the method is advantageous over methods using fixed nuclei since nucleic acids from the whole cell and not just from the nuclei can be barcoded. Moreover, encapsulating the cells in microcapsules prevents the cell loss that inadvertently occurs during split-pool barcoding techniques that are known in the art.

Further, the semi-permeable shell of the microcapsules allows the diffusion of low molecular weight compounds across shell while retaining the higher molecular weight nucleic acids within the microcapsule. This permits the exchange of reagents, enzymes, substrates etc during the method, such that each individual reaction of the barcoding method can be performed under optimal conditions. For example, harsh cell lysis conditions, followed by optimal conditions for a reverse transcriptase (RT) reaction, followed by optimal conditions for an oligonucleotide ligation reaction can be achieved, improving molecule capture and enzymatic reaction yields.

The method of barcoding of the invention can be applied to a wide range of single-cell nucleic acid analysis, including genome sequencing, and analysis of bacterial transcriptomics, proteomics, epigenomics, methylomics, and the non-coding transcriptome, and other application that rely on single-cell nucleic acid molecule barcoding.

Moreover, the method of barcoding of the invention has significant scalability such that it can match the processing capacity of the ultra-high through-put approaches of the prior art, allowing the barcoding of nucleic acid molecules from hundreds to millions of single cells at once, ensuring the capture of even the rarest cell types in a biological sample, and enabling efficient processing of clinical samples.

Further advantages of the products and methods of the invention will become apparent from the description that follows.

### BRIEF DESCRIPTION OF DRAWINGS

To assist understanding of the present disclosure and to show how embodiments may be put into effect, reference is made by way of example to the accompanying drawings described below. It should be noted that in the schematics provided various aspects are not drawn to scale.

FIG. 1. Schematics of an example of cell compartmentalization and processing for single-cell sequencing. Cells are encapsulated in an aqueous two-phase system (water-in oil) droplets using a microfluidics device. The water-in-oil droplets having a liquid core and a liquid shell are converted to microcapsules by polymerizing the shell. The encapsulated cells are lysed to release their internal content. The nuclei acid molecules released from the cell, such as encoding transcriptome and/or genome and/or epigenome are fragmented and subjected to barcoding procedure. The barcoding is performed using combinatorial indexing also known as a split-and-pool approach. The barcoded nucleic acid molecules are released from the microcapsules by breaking them, then the barcoded nucleic acid molecules are pooled and the DNA library is prepared for sequencing. 1—cell encapsulation 2—microcapsule generation, 3—lysis and further processing of encapsulated cells, 4—nucleic acids released from lysed cells are processed according to the final application such as mRNA is converted to cDNA, genome and epigenome is fragmented using enzymatic, chemical or physical tools, 5—the fragmented DNA is barcoded along with cDNA by attaching indexes to either 3′ or 5′ end of nucleic acids, barcoded DNA is released from compartments DNA library is prepared and sequenced.

FIG. 2. Agarose gel showing retention of DNA fragments inside the microcapsules. (FIG. 2A) GeneRuler 100 bp Plus DNA ladder (cat no. SM0321, ThermoFisherScientific) was encapsulated in aqueous two-phase system (water-in-oil) droplets and processed as follow: 1) The encapsulated DNA ladder was released immediately after droplet collection off-chip showing that there is no preferential DNA fragment loss during encapsulation process. 2) Microcapsules released into aqueous buffer at 4° C. retain encapsulated DNA fragments and shown now preferential loss. L—Ladder (GeneRuler 100 bp Plus DNA ladder), (FIG. 2B) The microcapsules from panel-A were subjected to photo-polymerization and one fraction of the microcapsules was then incubated at room temperature (3) and another fraction was incubated at 50° C. for 30 min (4). After the incubation microcapsules were broken, the released material was combined with DNA loading dye and sample was loaded in agarose wells for DNA electrophoresis. Arrow indicates the low molecular weight DNA fragments that were not retained in microcapsules during incubation at room, or higher temperature.

FIG. 3. Example experimental strategy for performing transcriptomics barcoding and sequencing of mRNA from individual cells. The individual cells encapsulated in microcapsules are lysed and their genomic DNA is digested by hydrolase (e.g. DNAse I). Next, the mRNA molecules are converted to cDNA by reverse transcription and poly(T) primers. The barcoding of cDNA is preferably performed using a combinatorial indexing strategy also known as split-and-pool approach. The barcoded nucleic acid molecules are released from compartments by breaking them, then the barcoded nucleic acid molecules are pooled and DNA library is prepared for sequencing. 1—cell lysis and gDNA digestion, 2—cDNA synthesis by reverse transcription, 3—combinatorial indexing of cDNA, 4—barcoded cDNA release from microcapsules, library preparation and sequencing.

FIG. 4. Example experimental strategy for simultaneous single-cell transcriptome and epigenome barcoding using microcapsules. The individual cells encapsulated in microcapsules are lysed and their genomic DNA is tagmented with Tn5 enzyme. The mRNA molecules are converted to cDNA by reverse transcription using poly(T) primers. The barcoding of cDNA and tagmented DNA is performed using a combinatorial indexing strategy also known as split-and-pool approach. The barcoded nucleic acid molecules are released from microcapsules by breaking them, then the barcoded nucleic acid molecules are pooled and DNA library is prepared for sequencing. 1—genomic DNA fragmentation by transposase, 2—cDNA synthesis by reverse transcription, 3—combinatorial indexing of cDNA and tagmented DNA, 4—the barcoded nucleic acid molecule release from microcapsules, library preparation and sequencing.

FIG. 5. Example experimental strategy for simultaneous profiling of transcriptome, chromatin and epigenetic modifications of single-cells. The single-cells encapsulated in microcapsules are subjected to series of multi-step reactions to i) profile open chromatin via Tn5 tagmentation, ii) profile histone modification and/or epigenetic factors through antibody assisted Tn5 tagmentation, iii) profile transcriptome via RT reaction, and iv) profile genome and DNA methylome via TET-assisted Pyridine Borane Sequencing (TAPS). Barcoding of nuclei acids is based on combinatorial indexing. The barcoded nucleic acid molecules are released from microcapsules by breaking them, then the barcoded nucleic acid molecules are pooled and DNA library is prepared for sequencing. 1—Tagmentation of chromatin DNA, 2—Tagmentation of DNA adjacent to epigenetic factors, 3—cDNA synthesis by reverse transcription, 4—combinatorial indexing, 5—Barcoded material release from microcapsules, 6—Conversion of 5-methyl cytosine to dihydrouracil by TAPS, 7—DNA library preparation and sequencing.

FIG. 6. Example experimental strategy for single-cell genome and methylome sequencing. The single-cells encapsulated in microcapsules are subjected to series of multi-step procedures during which the cells are lysed and their genome are fragmented. Fragmentation can be performed using physical (e.g. ultrasound), chemical (e.g. hydroxyl radicals) or enzymatic (endonucleases, transposase) means. The DNA fragments are barcoded following combinatorial indexing (split-pool) protocol. The barcoded nucleic acid molecules are released from compartments by breaking them, then the barcoded nucleic acid molecules are pooled and DNA library is prepared for sequencing. 1—Lysis of encapsulated cell, 2—genomic DNA fragmentation, 3—combinatorial indexing, 4—Barcoded material release from microcapsules, library preparation and sequencing, 5—Barcoded material release from microcapsules, 6—Conversion of 5-methyl cytosine to dihydrouracil by TAPS followed by library preparation and sequencing.

FIG. 7. Example experimental strategies for split-pool method for barcoding nucleic acids. A) Schematics of the 96-well plates where each well contains a primer with a unique barcode. B) Schematics of split-pool strategy for barcoding (indexing) the cDNA within the microcapsule. C) Schematics of split-pool strategy for barcoding (indexing) the fragmented DNA, within the microcapsule. The barcodes are marked as horizontal bars of different colour and labelled BC1, BC2 and BC3. The linker regions connecting the barcodes are coloured in grey. The barcodes BC1, BC2 and BC3 are originating from the corresponding plates indicated in panel A. The P7 and P5 indicate sequencing primers that anneal to Illumina's sequencing flow cell, UMI—unique molecular identifier. Tag—indicates a unique sequence that is part of the RT primer. Adapter 1 and/or Adapter 2 contains a unique sequence (index) that is specific to fragmentation reaction product.

FIG. 8. Mammalian cell encapsulation in GMA/dextran capsules. (A) Still photograph of the microfluidics device during GMA/dextran capsule generation. 1—a microchannel with an aqueous phase enriched in shell forming compound (GMA, gelatin methacrylate); 2—a microchannel with an aqueous phase enrich in core-forming compound (dextran); 3—carrier oil, 4—cell. (B) The water-in-oil droplets are converted to (C) micro-capsules through gelation and cross-linking. The gelation should be understood as a process during which the liquid-shell is converted into the solidified shell. The cross-linking should be understood as new covalent bond formation between two or more molecules. 5—shell enriched in GMA, 6—core enriched in dextran, 7—temperature-induced gelation, 8—covalent cross-linking, 9—semi-permeable shell composed of polymerized GMA. Scale bars, 100 μm.

FIG. 9. Capsule generation using gelatin with a different degree of methacrylate substitution. Capsules were generated using gelatin/dextran blend where gelatin contained different percentage of methacrylate substitution. For each test 3% (w/v) of gelatin polymer with of a given degree of substitution, and 15% (w/v) dextran (MW˜500 k) were used. (A) gelatin with 0% degree of substitution, (B) GMA with 40% degree of substitution, (C) GMA with 60% degree of substitution, (D) GMA with 80% degree of substitution. Scale bars, 100 μm.

FIG. 10. Capsule generation using GMA with a low-degree of substitution. Capsules were generated using 5% (w/v) GMA with 40% degree of substitution and 15% (w/v) dextran (MW˜500 k). Scale bar, 100 μm.

FIG. 11. Capsule generation using different polymerization approaches. (A) Capsule generation process where cross-linking of the capsule shell was performed during droplet generation step by exposing liquid droplets to photo-illumination. (B) Capsule generation process where cross-linking of the capsule shell was performed by exposing off-chip collected emulsion to photo-illumination. (C) Capsule generation process where at first the capsules' shell was solidified during temperature-induced gelation process, and only then cross-linked by photo-illumination. (D) Capsules, where the shell was polymerized following emulsion collection off-chip, incubation at 4° C. to induce gelation of the shell, dispersed solidified capsules in aqueous buffer only then cross-linked via light-induced polymerization. 1—photo-illumination, 2—capsule collection off-chip, 3—carrier oil, 4—liquid core, 5—chemically cross-linked shell, 6—capsule dispersion in aqueous buffer, 7—water phase, 8—generation of water-in-oil droplets, 9—liquid shell, 10—droplet incubation at low temperature (e.g. 4° C.) to induce the solidification of the shell, 11—solidified shell.

FIG. 12. Capsule generation using temperature-induced and/or light-induced polymerization. Photographs show capsules dispersed in aqueous buffer after polymerization of capsules' shell by temperature-induced gelation and/or light-induced cross-linking. (A) Capsules were generated by cross-linking capsule shell during droplet generation step by exposing droplets to photo-illumination and then dispersed in an aqueous buffer. (B) Capsules, where the shell was polymerized by photo-illumination immediately after emulsion collection off-chip and then dispersed in an aqueous buffer. (C) Capsules, where the shell was polymerized following emulsion collected off-chip, incubation at 4° C. to induce gelation (solidification) of the shell and cross-linking via light-induced polymerization, and then dispersed in an aqueous buffer. (D) Capsules, where the shell was polymerized following emulsion collection off-chip, incubation at 4° C. to induce gelation of the shell, dispersing capsules in an aqueous phase and only then cross-linking via light-induced polymerization. Scale bars, 100 μm.

FIG. 13. Capsule generation using chemical agent-induced polymerization. Photographs show capsules dispersed in an aqueous buffer after polymerization of capsules' shell by chemical agent induced cross-linking and the combination of temperature-induced gelation and chemical agent induced cross-linking. (A) Capsules were generated using GMA/dextran blend where GMA phase was supplemented with 0.3% (w/v) APS, and carrier oil phase was supplemented with 0.4% (v/v) TEMED. The emulsion was collected off-chip and incubated at room temperature for 2 hours to allow sufficient period of time for shell polymerization to occur. Shown are the resulting capsules suspended in an aqueous buffer. (B) The water-in-oil droplets containing GMA/dextran blend were generated and collected off-chip, and incubated at 4° C. for 30 min to induce solidification of the shell. The solidified capsules were then resuspended in an aqueous buffer containing polymerization initiators (0.3% (w/v) APS and 0.4% (v/v) TEMED) and incubated at room temperature for 2 h to induce chemical cross-linking of capsules' shell. Shown are the resulting capsules suspended in an aqueous buffer. Scale bars, 100 μm.

FIG. 14. Capsule production using polyhydroxy compounds. Capsules were generated using a mixture of GMA and polyhydroxy compounds. (A) Capsules composed of GMA and hydroxyethyl-cellulose, solidified and polymerized at ˜4° C. temperature. (B) Capsules composed of GMA and Ficoll PM400, solidified and polymerized at ˜4° C. temperature. Scale bars, 100 μm.

FIG. 15. Capsule production using ion liquids. Capsules were generated using a mixture of GMA and ammonium sulfate. Scale bar, 100 μm.

FIG. 16. Generation of capsules having different diameter. (A) Capsules having a diameter of 35 μm, (B) Capsules having a diameter of 60 μm, (C) Capsules having a diameter of 180 μm, (D) Capsules having a diameter of 24 μm. Scales bars, 100 μm.

FIG. 17. Capsule size control by temperature. (A) Capsules composed of GMA and dextran, were solidified and photo-polymerized at ˜4° C. temperature. (B) Capsules composed of GMA and dextran, were solidified at ˜4° C. temperature and photo-polymerized after 15 minutes incubation at room (˜22° C.) temperature. (C) Capsules composed of GMA and hydroxyethyl-cellulose, were solidified and photo-polymerized at ˜4° C. temperature. (D) Capsules composed of GMA and hydroxyethyl-cellulose, were solidified at ˜4° C. temperature and photo-polymerized after 15 minutes incubation at room (˜22° C.) temperature. (E) Capsules composed of GMA and Ficoll PM400, were solidified and photo-polymerized at ˜4° C. temperature. (F) Capsules composed of GMA and Ficoll PM400, were solidified at ˜4° C. temperature and photo-polymerized after 15 minutes incubation at room (˜22° C.) temperature. Scale bars, 100 μm.

FIG. 18. Increasing the concentration of the shell-forming precursor leads to capsules with thicker shells. The capsules were generated by emulsifying 5% (w/v) GMA with 15% (w/v) dextran solutions followed by physical gelation and light-induced cross-linking of the shell either at ˜4° C. (panel A) or ˜22° C. temperature (panel B). (A) The photograph shows ˜68 μm diameter capsules having 6.5 μm shell and 55 μm core. (B) The photograph shows ˜82 μm diameter capsules having 6 μm shell and 70 μm core. Scale bars, 100 μm.

FIG. 19. Single-cell isolation in capsules. Cell retention comparison in droplets and in capsules. Boxplots (FIG. 17A) and bar plots (FIG. 17B) representing mammalian cell retention in droplets and capsules. Independent samples t-test showed there is no statistically significant difference of cell occupancy in droplets and capsules. (p=0.2281). Cell occupancy in droplets is 8.2±1.6, cell occupancy in capsules is 8.8±1.3. In both FIG. 19A and FIG. 19B values for retention in droplets is shown on left with the values for retention in capsules shown on the right.

FIG. 20. Epifluorescence microscopy analysis of capsules after multiplex RT-PCR. The first (top) row shows multiplex RT-PCR results on capsules carrying a mixture of K562 and HEK293 cells. The second row shows multiplex RT-PCR results on capsules carrying K562 cells. The third row shows multiplex RT-PCR results on capsules carrying HEK293 cells. The fourth row shows multiplex RT-PCR results on capsules carrying a mixture of K562 and HEK293 cells, when reverse transcription enzyme was omitted from reaction mix (no RT). The fifth row shows multiplex RT-PCR results on capsules carrying no template. The first column shows bright field images. The second column shows Alexa Fluor 647 dye fluorescene images corresponding to ACTB positive capsules. The third column shows Alexa Fluor 488 dye fluorescene images corresponding to PTPRC positive capsules. The fourth column shows Alexa Fluor 555 dye fluorescene images corresponding to YAP positive capsules. Fifth column shows merged images demonstrating cell specific markers (PTPRC and YAP) overlapping with ACTB expression. The ACTB positive capsule lacking PTPRC and YAP signal indicate ambient ACTB transcript levels originating from the lysed cells. Scale bars, 100 μm.

### DETAILED DESCRIPTION OF THE INVENTION

Throughout the text the terms of ‘comprising’ and ‘containing’ have been used interchangeably and have the same meaning.

The terms “approximately” or “about” are used herein and generally refer to a range of ±30% of the stated value, preferably ±20% of the stated value, more preferably ±10% of the stated value, and even more preferably ±5% of the stated value.

Articles such as “a,” “an,” and “the” include the singular and the plural reference unless the context clearly indicates otherwise. For example, a reference to “a cell” includes one cell and two or more of such cells.

As described above, the present invention relates a method for sequentially attaching a plurality of oligonucleotides to nucleic acids to produce barcoded nucleic acids, wherein the nucleic acids are obtained from a plurality of cells, wherein the plurality of cells are in a plurality of microcapsules, each microcapsule comprising a semi-permeable shell and a core, wherein each cell is in a separate microcapsule, the method comprising:
(a) lysing the cells within the microcapsules to release DNA and RNA inside the microcapsules;
(b) optionally in the microcapsule:
   (i) converting released cellular RNA to cDNA by reverse transcription; and/or
   (ii) fragmenting the released cellular DNA to produce fragmented DNA; and/or
   (iii) amplifying fragmented DNA and/or cDNA,
(c) applying a combinatorial indexing strategy to attach the plurality of oligonucleotides one at a time to the released cellular DNA, the released cellular RNA, the cDNA, and/or the fragmented DNA and/or amplified DNA and/or cDNA in each microcapsule to produce barcoded nucleic acids;
wherein the barcoded nucleic acids produced in each microcapsule comprise a sequence of oligonucleotides that is specific to that microcapsule.

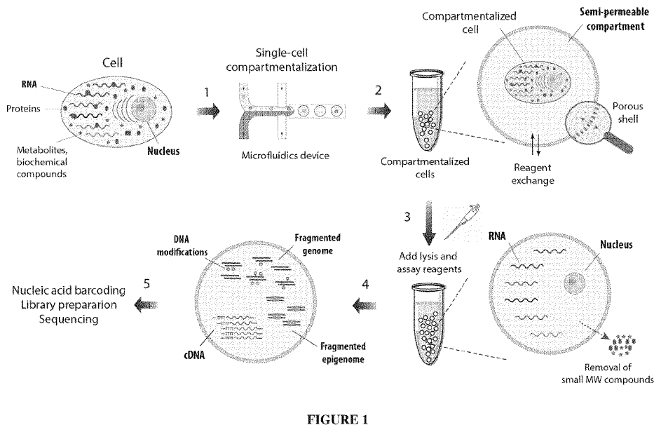

As shown in FIG. 1, individual cells are contained within microcapsules. The semi-permeable (porous) shell of the microcapsule retains the cell and the nucleic acids released from the cell by lysis in (a), i.e. the released cellular DNA (nuclear and mitochondrial) and the released cellular RNA, inside the microcapsule while allowing reagents, enzymes, substrates, oligonucleotides, etc for the performance of (a) to (c) to diffuse into and out of the core of the microcapsule. Microcapsules suitable for the performance of the invention are discussed further below.

As a result of using cells encapsulated in microcapsules with a semi-permeable shell, cell lysis can be performed under conditions that would normally be incompatible with combinatorial indexing/split-and-pool synthesis. For example, in the method of the invention the microcapsules can be contacted with detergents, and/or guanidinium chloride to lyse the cells within the microcapsules. The microcapsules can then be subjected to washing (buffer/reagent exchange) in order to remove the lysis reagents and cell-lysis products from the microcapsules while retaining the released nucleic acids (DNA and RNA) within the microcapsule. In this manner, any lysis reagents that would be incompatible with the combinatorial indexing/split-and-pool synthesis can be removed before (b) and (c). Moreover, the microcapsules described herein are thermostable and can be heated (at 70° C., or up to 98° C.) without disintegrating. Thermal denaturation prior (b) and (c) may improve cell lysis, denaturation biomolecules (e.g. proteins), and/or melting of nucleic acids strands.

The method of the present invention uses combinatorial indexing also known as split-and-pool synthesis to attach a succession of oligonucleotides to the DNA molecules obtained from the cell, while the DNA molecules are retained within the microcapsules, to build up a barcode (comprising the succession of oligonucleotides) on each DNA molecule that is unique to the cell from which it originates, i.e. all the barcoded DNA molecules that are obtained from a single cell have the same barcode, the barcode being different from that of DNA molecules obtained from different cells. The barcode means that the cell from which the barcoded DNA molecules can always be grouped with other barcoded DNA molecules from the same cell, even after the barcoded DNA molecules from different cells are combined.

In particular, the combinatorial indexing/split-and-pool synthesis of (c) may comprise:
(i) randomly distributing the plurality of microcapsules into a plurality of compartments, wherein each compartment comprises more than one microcapsule;
(ii) processing the microcapsules in each compartment to attach an oligonucleotide to the nucleic acid in the microcapsules, wherein a different oligonucleotide is utilised in each compartment;
(iii) pooling and randomly re-distributing the plurality of microcapsules into a further plurality of compartments, wherein each further compartment comprises more than one microcapsule, and processing the microcapsules in each compartment to attach a further oligonucleotide to the nucleic acid in the microcapsules, wherein a different oligonucleotide is utilised in each of the further compartments; and optionally repeating step (iii) one or more times.

The plurality of compartments may be laboratory tubes or wells on a microtitre plate, e.g. a 96-well microtitre plate.

In each iteration of the processing step the microcapsules in a particular compartment are contacted with a compartment-specific (e.g. well-specific) oligonucleotide. The oligonucleotides are attached to the DNA molecules in each microcapsule in that compartment (e.g. well). After each iteration of the processing step, all the microcapsules from all the compartments are pooled and re-split (re-distributed).

The method may comprise one or more washing steps after the oligonucleotides are attached to remove unused oligonucleotides from the microcapsules before they are pooled.

The method may comprise a step after each pooling where the microcapsules are mixed before being split/re-split (distributed/re-distributed), so as to ensure that in each splitting step the microcapsules are split between the compartments in a completely randomized manner.

The method may also comprise attaching a unique molecular identifier (UMI) to the DNA molecules obtained from the cell. A unique molecular identifier is a tag comprising a short random sequence. Each UMI has a unique sequence, such that individual DNA molecules can be identified. In a preferred scenario the UMI is a nucleotide sequence comprising random nucleotides from 4 to 24 long, and preferably random nucleotides between 6 and 16 long. The UMI can be utilised to increase sensitivity of variant detection and filter out variants produced during later PCR.

The oligonucleotides may be attached by a ligation reaction or attached via a nucleic acid extension reaction.

The barcoded DNA molecules can be released from the microcapsules by breaking the shell of the microcapsule. In particular, as discussed below, depending on the nature of the shell this can be broken down by chemical or enzymatic means, e.g. where the shell comprises peptide bonds the shell can be broken and the barcoded DNA released using suitable protease enzymes.

Once released from the microcapsules the barcoded DNA can be subjected to further processing and/or analysis steps. In particular the barcoded DNA can be purified, amplified, and/or sequenced. Preferably the barcoded DNA is used to produce a DNA library.

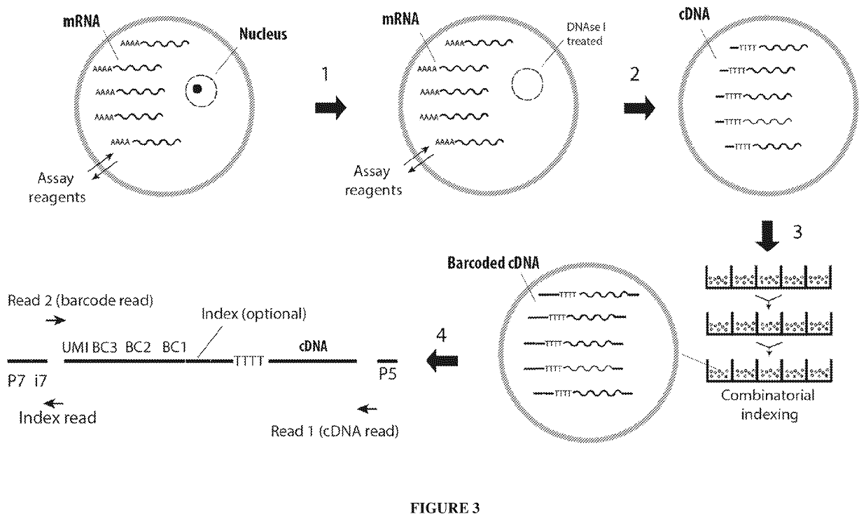

In one example of the method, (b) comprises converting mRNA molecules to DNA molecules by reverse transcription and combinatorial indexing in (c) to produce barcoded cDNA. In particular, as shown in FIG. 3, the microcapsule is optionally contacted with DNAse I, which diffuses into the microcapsule and cleaves the genomic DNA (and any mitochondrial DNA), leaving the mRNA and other RNA molecules intact and available to be converted into cDNA by reverse transcription and barcoded in (c).

The method may further comprise a step of fragmenting the DNA molecules, which have been released into the microcapsules by the cell lysis. In particular, such fragmentation may be performed by physical, chemical or enzymatic means e.g. using ultrasound, using complexes that generate hydroxyl radicals, using a transposase and/or a Tn5-antibody fusion protein.

### RNA Barcoding

In one example of the present invention, the method may be used to obtain barcoded DNA molecules that represent the transcriptome of a plurality of cells. As schematically shown in FIG. 3, in (b) of the method the microcapsules carrying transcriptomes of each individual cell are converted to copy DNA (cDNA). Next, the microcapsules are randomly distributed into, e.g. a 96-well plate, and well-specific oligonucleotides are ligated to 5′-end of each cDNA molecule. It should be understood the using appropriately modified oligonucleotides, e.g., having 5′ phosphate group, the ligation may be performed to 3′-end of each cDNA molecule. Next, the microcapsules from all wells are washed, pooled, and redistributed into a second 96-well plate, to initiate a second round of ligation. Additional round(s) of washing, pooling, redistribution into a second 96-well plate, and ligation may be performed until a desirable length barcodes are generated. Finally, an oligonucleotide containing a unique molecular identifier (UMI), or a further barcode comprising a UMI, is appended with a third round of pooling, splitting, and ligation (although it is also possible to add UMI during the first round of ligation and then attach barcodes to cDNA-UMI fragments). By repeating the process three times each cell will have one of 963=884,736 possible oligonucleotide combinations (i.e. barcodes), while repeating it three times in 384-plates, will generate 384³˜56 million barcodes. We have previously reported similar split-and-pool method for synthesizing barcoded beads for scRNA-Seq applications [37]. At any step in the protocol sequencing adapters may be added, and in a preferred scenario at the final steps of split-pool process the sequencing adapters are added during a template switching reaction. The resulting barcoded DNA molecules are amplified by PCR (fragmented if needed according the protocol), purified and sequenced. After sequencing, each transcriptome is assembled by combining the reads containing the same barcode combinations.

### Epigenome Barcoding

In another example of the present invention, the method may be used as part of a method of epigenome barcoding. In particular, if single-cell RNA sequencing can reveal the transcriptional state of a cell at a given time point, it provides little insight into the epigenome. Profiling the accessible chromatin in individual cells can reveal regulatory genomic sequences that have important role in gene expression and thereby cell phenotype [38-40]. Although mapping of open chromatin and transcriptome individually in single cells either one at a time can provide interesting biological insights [4-8], only by simultaneously profiling of both, mRNA and chromatin structure within the same cells, we can establish a direct link between the regulatory cis-/trans-factors and transcriptional output. Most recent developments have moved towards this direction by introducing assays for performing joined-profiling of transcriptome and open chromatin state [17-19]. However, critically, in all high-throughput platforms reported to-date the reliance of joined-profiling on cell nuclei instead of whole cells means that sequencing data obtained reveals only a limited set of transcripts that were present in the nuclei at a given moment of cell isolation from tissue. Even though nuclear mRNA fraction can be used to identify cell types [41, 42], such information does not provide enough granularity and precision needed for deeper insights of complex biological processes.

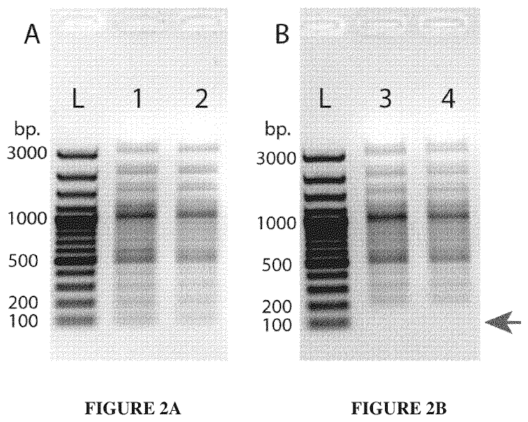

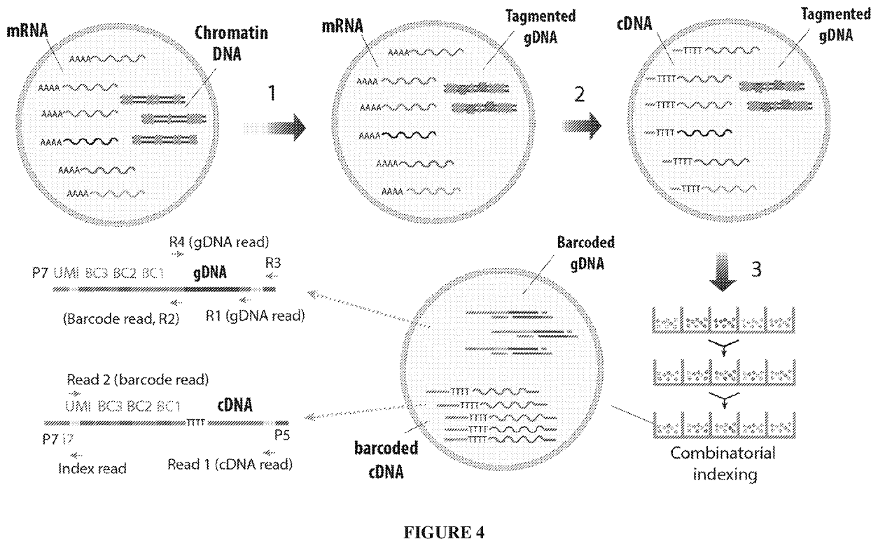

An example of the use of the method of present invention in a method of simultaneous epigenome and transcriptome barcoding is shown in FIG. 4. In particular, at first single cells isolated in microcapsules are lysed. Then the microcapsules are washed in aqueous buffer to replace the lysis reagents and/or remove inhibitory lysis products. The microcapsules are dispersed in another reaction buffer containing assay reagents to initiate a transposition reaction. Because DNA fragments generated by Tn5 transposase (taggmented) have size distribution above 200 bp [5, 7], they are effectively retained inside the microcapsules (FIG. 2), while other reaction components and short DNA primers/oligonucleotides passively move between the microcapsule core and the external environment. The taggmentation reaction is first carried out on cell lysate (encapsulated inside the microcapsules) with Tn5 transposase containing well-specific adaptors that comprise a first well-specific oligonucleotide as the first part of the barcode. After taggmentation of the genomic DNA (gDNA), the microcapsules are resuspended in another reaction mix to initiate cDNA synthesis from the mRNA. The cells are subjected to RT reaction using polyT primers containing the same first well-specific oligonucleotide, so that the chromatin DNA fragments and cDNA from the same well are labelled with the same first-round oligonucleotides. Once cDNA is generated and gDNA is fragmented by Tn5 activity the microcapsules are subjected to a combinatorial indexing to simultaneously create barcodes on both (i) the open chromatin fragments generated by the Tn5 transposase, and (ii) the cDNA molecules generated during reverse transcription reaction. To perform combinatorial indexing the microcapsules are pooled and redistributed to a microtiter plate containing well-specific DNA oligonucleotides, which are ligated to 5′ ends of the taggmented DNA fragments and the cDNA molecules. Repeating the ligation procedure two more times after pooling and splitting, the combinatorial diversity of more than 10∂unique barcode sequences can be achieved. The barcoded DNA may be amplified, split into two portions and digested with restriction enzymes targeting the pre-designed recognition sites in Tn5 and RT primers, that way enriching DNA and RNA libraries for next-generation sequencing (NGS).

The methods disclosed herein address several critical limitations of the prior art methods:
First, if current state-of-the-art droplet-microfluidics and split-pool methods rely on cell nuclei as an input material, the approach revealed here is based on whole cells, which can be critical for identifying novel gene targets and regulatory functions of the genome elements.
Second, the transposition and reverse transcription reactions are performed independently from each other, under optimal conditions and on the lysed cells, which is impossible to do using current droplet-microfluidic technologies.
Third, the cells in the microcapsules can be treated with chemicals and bioreagents that are incompatible with current protocols. For example, as it was reported in the original work the post-tagmented Tn5 enzyme removal from the genome is critical for high mapping efficiency, yet it requires SDS [43], a detergent that is incompatible with existing droplet-based platforms. In addition, there are multiple compounds available that enhance nuclei permeability and thus increase transposition reaction efficiency (e.g. one of them is based on bromobenzylidene-naphthalene-sulfonamide [44]).

### Epigenome, Genome and Transcriptome Barcoding

The integration of the different layers of genetic information from thousands and millions of individual cells can offer us a unique opportunity to link multiple aspects of cellular identity. If early efforts of performing multi-omics assays were restricted to analysis of a few dozen of sorted cells [30, 33, 35], the rise of cellular barcoding strategies opened new possibilities to analyze multiple-omics modalities in thousands of single-cells, and more [45]. For example, using droplet-based approaches, several groups have combined scRNA-Seq and CRISPR-Cas9 methodologies to investigate the effects of different genetic perturbations on gene expression [46], multimodal immunophenotyping of cancer cells [47] or antigen profiling [48]. Existing combinatorial single-cell indexing platforms, based on split-and-pool barcoding strategies, offers alternative paths of perform multimodal analysis [17], however, at the cost of reduced sensitivity and undesirable cell loss during sample preparation.

In one example, the method of the invention can be used to combine scRNA-Seq [24], scATAC-Seq [6], CUT&Tag [49] (or CUT&RUN [50]) and bisulfite-free DNA methylation sequencing [51] methodologies to perform an integrated and high-throughput multi-omics analysis of single-cells. Critically, the cells have to be isolated to microcapsules before processing them through aforementioned methodologies. The compartmentalization of cells in microcapsules provides numerous analytical advantages, including enabling removal of intracellular inhibitors, allowing the performance of multi-step reaction on the same cell, and allowing the cells to be subjected to biochemical reaction conditions that are incompatible with existing methodologies.

As shown in FIG. 5, the encapsulated cells can be subjected to transposase-driven DNA fragmentation reaction (taggmentation). During taggmentation the open (accessible) chromatin regions are fragmented by transposase, which catalyzes insertion of the DNA adapters. These adapters can comprise oligonucleotides as a basis for a barcode. In addition, the specific epigenetic marks and regulatory proteins from the same cells can also be targeted, by Tn5 fused to target specific antibody. Upon binding the DNA sequence close to the target histone modification under antibody guidance, the Tn5 transposase fused to protein-A inserts the adapter. To distinguish double stranded DNA (dsDNA) ends generated during CUT&Tag approach Tn5 protein can be preloaded with unique indexed adapters. For example, using 11 unique indexes and target antibodies the said CUT&Tag approach can be used to multiplex all 11 histone modifications that are known to exist in mammalian cells. After taggmentation of the genomic DNA (gDNA), the microcapsules are resuspended in another reaction mix to initiate cDNA synthesis from the mRNA. Once the open chromatin and DNA surrounding targets of interest (e.g. modified histones, transcription factors) are tagged with unique indexes the encapsulated cells are subjected to RT reaction. Following these steps, the microcapsules will contain material derived from a single cell corresponding to: i) transcriptome (in the form of cDNA), ii) open chromatin (in the form of indexed Tn5 tagmented DNA) and iii) DNA regulatory elements (in the form of indexed CUT&Tag tagmented DNA). In one example, the cDNA and gDNA fragments are barcoded using a ligation-based combinatorial indexing strategy to simultaneously tag all nucleic acid fragments generated by the Tn5 transposases and the cDNA molecules generated during RT. In another example, the fragmented gDNA is treated with TET/pyridine borane for bisulfite-free direct detection of methylated cytosines, 5-mC [51]. The semi-permeable nature of shell of the microcapsules enables chemical and enzymatic treatment of nuclei acids, while retaining them inside. The post-TET/pyridine borane treated DNA fragments can then be barcoded using a ligation-based combinatorial indexing strategy to simultaneously tag all nucleic acid fragments generated by the Tn5 transposases and the cDNA molecules generated during RT.

### Genome Barcoding and Sequencing

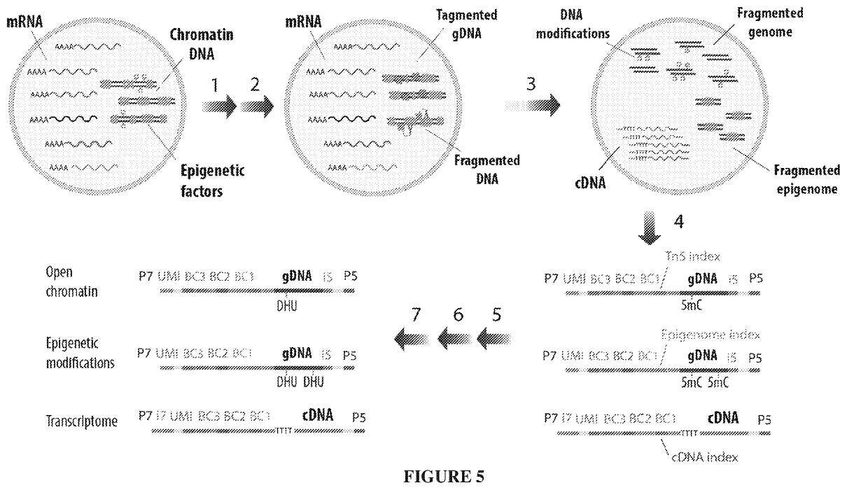

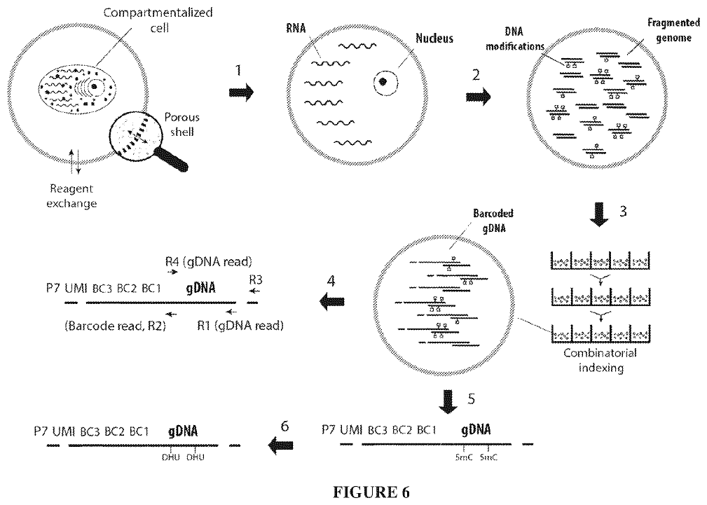

In another example (as shown in FIG. 6), the cells isolated in the microcapsules are subject to multi-step reactions to perform genomic DNA fragmentation and barcoding for next-generation sequencing. The cells compartmentalised in microcapsules are lysed and their genomic DNA is fragmented using enzymatic (endonucleases, transposase), physical (e.g. ultrasound) or chemical means (e.g. complexes that generate hydroxyl radicals, such as iron-EDTA, can be used to introduce random DNA cleavage). The fragmented genome can then be barcoded using combinatorial indexing (split-and-pool approach) as described above. The barcoded DNA fragments can then be purified, converted to DNA library by adding sequencing adapters and sequenced.

### DNA Methylome Barcoding and Sequencing

In another example (FIG. 6), the cells isolated in microcapsules are subject to multi-step reactions to perform DNA methylome barcoding for next-generation sequencing. Similar to “Genome Barcoding and Sequencing” above, the cells compartmentalised in microcapsules are lysed and their genomic DNA is fragmented using enzymatic (endonucleases, transposase), physical (e.g. ultrasound) or chemical means (e.g. complexes that generate hydroxyl radicals, such as iron-EDTA, can be used to introduce random DNA cleavage). The fragmented genome can then be barcoded using the combinatorial indexing (split-and-pool approach) described above. Alternatively, the microcapsules containing the fragmented DNA can be re-encapsulated in another droplet having barcoded DNA oligonucleotides and either ligated to DNA fragments or extended by DNA polymerase. The barcoded DNA fragments can then be applied to bisulfite conversion, a process in which the deamination of unmethylated cytosines into uracils occurs, while methylated cytosines (both 5-methylcytosine and 5-hydroxymethylcytosine) remain unchanged. Alternatively, the fragmented DNA can be subjected to TET/pyridine borane treatment [51]. The post-TET/pyridine borane treated DNA fragments can be purified, converted to DNA library by adding sequencing adapters and sequenced.

### Microcapsules

As described above, the microcapsules to be used in the present invention have a semi-permeable shell and a core. The semi-permeable shell of the microcapsule retains the cell and the nucleic acids released from the cell by lysis in (a), i.e. the DNA (nuclear and mitochondrial), the mRNA and some other RNAs, inside the microcapsule while allowing reagents, enzymes, oligonucleotides, salts, substrates, etc for the performance of (a) to (c) to diffuse into and out of the core of the microcapsule. For example, the permeability of the shell can be chosen so as to ensure that the smaller oligonucleotides used in the combinatorial indexing/split-and-pool synthesis can diffuse into the core of the microcapsule (and unused oligonucleotides can diffuse out again during the washing steps), while the DNA molecules obtained from the cell are retained inside microcapsule and do not diffuse out.

In general, the semi-permeable shell allows for the diffusion of smaller molecular weight compounds of approximately MW 200,000 or less through the shell, while retaining larger molecular weight compounds of approximately MW 300,000 and above. For example, as shown in FIG. 2, in some examples the double-stranded DNA of 200 nucleotides and larger are retained inside the microcapsule, while smaller DNA fragments (˜100 bp or less) diffuse out.

In general the microcapsules have high circularity and high concentricity. Considering the average radius, R, of the microcapsule being R=√(square root over (S/π)), wherein S is the equatorial transverse surface of the capsule. The circularity, C, is a ratio of the minor axis (R min) over the major axis (R max) of the ellipse adjusted to the external edge of the projected equatorial section. In the present disclosure the microcapsule may have a circularity C=0.8±0.2, preferably C=0.9±0.1, more preferably C=0.94±0.06, and even more preferably C=0.95±0.05.

The concentricity, O, of the microcapsule is defined as O=(Wmin/Wmax)*100%, wherein Wmin is thinnest part of the shell and Wmax is the thickest part of the shell. In the present disclosure the microcapsule shows O≥66%. The high circularity and concentricity of microcapsules may be advantageous during the performance of reactions in the microcapsule to ensure that reactions are efficient.

The semi-permeable shell of the microcapsule may comprise a gel formed from a polymer, wherein the polymer in the gel is covalently cross-linked. In particular, the polymer is a polyampholyte and/or a polyelectrolyte or a synthetic polymer. The term ‘polyampholyte’ refers to a polyelectrolyte that bears both cationic and anionic groups, or corresponding ionizable groups, and where the ‘polyelectrolytes’ are polymers whose repeating units bear an electrolyte group. It should be understood that term ‘polyampholyte’ and ‘ampholytic polymer’ are synonyms as defined by IUPAC.

In particular, the gel may be formed from a polyampholyte and/or a polyelectrolyte that comprise a covalently cross-linkable group, or may be formed from a polyampholyte and/or a polyelectrolyte, or a synthetic polymer that is modified with a chemical group, which chemical group participates in the covalent cross-link. The chemical group may be selected from the group consisting of acrydite, acrylate, methacryloyl, acrylamide, methacrylamide, bisacrylamide, methacrylate, methacrylic acid, acrylic acid, polyacrylic acid, methacrylic anhydride, acryloyl, vinyl, vinylsulfone, vinylpyrrolidone, thiol, disulphide, cystamine, carboxyl, amine, imine, azide, triazole, tetrazine, azidophenylalanine, alkynyl, alkenyl, alkynes, diisocyanate, hydroxypropionic acid, hydroxy phenol, azobenzene, methylcyclopropene, trans-cyclooctene (TCO), norbornene, diacrylcyclooctyne (DBCO) or cyclooctanyl moieties and/or reagents.

The polymer may comprise amino acids and be a peptide, a polypeptide, an oligopeptide or a protein. Accordingly, the polymer may be described as “proteinaceous”. In particular, where the polyampholyte is a protein, polypeptides or oligopeptide, the primary amino acid sequence may comprise at least 10% disorder promoting amino acids, and preferably at least 30%. Disorder promoting amino acids include proline, glycine, glutamic acid/glutamate, serine, lysine, alanine, arginine, and glutamine. Without wishing to be bound by theory it is considered that such disorder promoting amino acids also promote liquid-liquid phase separation in the droplet during formation of the microcapsule (which is discussed further below). Furthermore, the proteinaceous polyampholytes that show liquid-liquid phase separation properties are often characterized by long segments of low diversity amino acids. These segments are often repetitive and are enriched in glycine (G), glutamine (Q), asparagine (N), serine (S), arginine (R), lysine (K), aspartate (D), glutamate (E) or aromatic amino acids such as phenylalanine (F) and tyrosine (Y) amino acids. These segments often encompass multiple short motifs such as YG/S-, FG-, RG-, GY-, KSPEA-, SY- and Q/N-rich regions, or regions of alternating charges [52].

The polyampholyte may be one that is capable of forming a coacervate in response to salts, temperature change, pH change or ionic change of a solvent in which the said polyampholyte is present during formation of the semi-permeable shell.

In preferred embodiments the polyampholyte and/or the polyelectrolyte is a “thermo-responsive” polymer capable of forming a gel in response to a temperature change, for example when cooled, below sol-gel transition temperature. The gel that is formed in response to the temperature change is a mesh or 3-dimensional network of polymer strands, with a solid structure due to physical cross-linking of individual polymer strands.

For example, the polyampholyte may be selected from the group consisting of collagen, mucin, laminin, elastin, elastin-like polypeptides, fibrin, silk fibrion, fibronectin, vimentin, glycinin, gluten, casein, or hydrolyzed forms thereof, such as gelatin.

Particularly preferred is a polyampholyte selected from gelatin, gelatin methacryloyl, gelatin methacrylamide, gelatin acrylamide and gelatin methacrylate, and preferably wherein the polyampholyte is gelatin methyacrylate.

The core of the microcapsule may comprise an antichaotropic agent and/or a polyhydroxy compound. In particular, the antichaotropic agent may be kosmotropic salt, and in particular may be a carbonate, a sulphate, a phosphate or a citrate. In a preferred example kosmotropic salt is an ammonium sulphate.

The polyhydroxy compound may be a naturally occurring polymer or derivatives thereof. In particular, the polyhydroxy compound may be selected from a polysaccharide, a carbohydrate, an oligosaccharide, or a sugar, which can be natural or synthetic. In one example, the polyhydroxy compound is one or more of dextran, alginate, hyaluronic acid, glucan, glycogen, starch (amylose, amylopectin), agarose, agar-agar, heparin, pectin, cellulose (including hydroxyethyl cellulose), hemicellulose, chitosan, chitin, xanthan gum, curdian, pullulan, inulin, graminan, levan, carrageenan, polyglycerol, and derivatives of the foregoing that are chemically modified or partly hydrolyzed. Preferably the polyhydroxy compound is glucan, more preferably dextran. Alternatively, the polyhydroxy compound may be a synthetic polymer, such as Ficoll (e.g. Ficoll PM 4000).

The polyhydroxy compound may have a molecular weight of 300 Da to 5000 kDa. In one example the molecular weight is greater than 10 kDa (i.e. is between 10 kDa and 800 kDa). In another example the molecular weight is greater than 100 kDa (i.e. is between 100 kDa and 800 kDa). In a preferred example, the polyhydroxy compound has a molecular weight of 400 to 2000 kDa, more preferably approximately 500 kDa.

The plurality of cells for use in the method of the invention may be from any source and may be prokaryotic or eukaryotic. Preferably the cells are eukaryotic, preferably non-human mammalian or human cells. The cells may be obtained from a sample, e.g. a sample obtained from a human or animal body, such as a tissue sample. The cells may be labelled by Ab-DNA conjugates often known as cell hashing [15] prior to being used in the method of the invention.

The method may comprise a step of producing the plurality of cells in the plurality of microcapsules prior to (a) by encapsulation, i.e. forming a microcapsule around each cell.

In particular, this method may comprise:
(1) forming water-in-oil droplets comprising a cell, a first solute and a second solute, wherein the first solute comprises a polymer comprising one or more covalently cross-linkable groups,
(2) allowing aqueous phase separation inside the water-in-oil droplet into a shell phase enriched in the first solute and a core phase enriched in the second solute, and gelation and/or precipitation in the shell phase to form an intermediate microcapsule;
(3) forming intermolecular covalent cross-links with the one or more covalently cross-linkable groups to form the microcapsule comprising a semi-permeable shell of covalently cross-linked polymer and a core, wherein the cell is in the core of the microcapsule. In particular, the second solute may be the antichaotropic agent and/or a polyhydroxy compound.

In preferred cases, the polymer is a thermo-responsive polymer as described above. In such cases, step (2) may comprise changing the temperature of the water-in-oil droplet so as to induce physical cross-linking of the thermo-responsive polymer to achieve solidification in the shell phase to form the intermediate microcapsule, wherein the solidified gel is a thermoreversible gel. Changing the temperature may comprises cooling the water-in-oil droplet to a temperature between 4° C. and 30° C., and preferably to below 10° C.

The covalent cross-linking in (3) may comprise exposing the intermediate microcapsule to a chemical agent, irradiation, or heat, or any combination thereof, to covalently cross-link the polymer. Preferably, (3) comprises covalently cross-linking by photo-polymerisation.

Further methods of forming suitable microcapsules are described in US 2020/0400538 A1 (which describes encapsulation of cells in microcapsules made in particular from PEGDA and dextran), and provisional application U.S. 63/284,657. Moreover, as demonstrated in Example 1, suitable microcapsules can be made with a polyampholyte (gelatin) and a polysaccharide (dextran).

After encapsulation of cells in microcapsules, the microcapsules may be sorted so as to enrich for microcapsules comprising single cells and avoid the processing of microcapsules comprising more than one cell. In particular, the microcapsules may be sorted into a first pool of microcapsules comprising one cell and a second pool of microcapsules comprising more than one cell and/or no cells. (Microcapsules comprising no cells, and/or having more than once cell can be discarded from combinatorial indexing steps.) Sorting may be performed using a fluorescence-activated cell sorter (FACS). By enriching for microcapsules comprising only single cells at the start of the method, the quality of the ultimate data obtained from the method can be improved since data sets relating to cell doublets or multiplets can be reduced as far as possible.

The examples which follow are to be understood as illustrative examples of embodiments of the invention. Further embodiments and examples are envisaged. Any feature described in relation to any one example or embodiment may also be used in combination with one or more features of any other of the examples or embodiments, or any combination of any other of the examples or embodiments, except where the circumstances dictate otherwise. Furthermore, equivalents and modifications not described herein may also be employed within the scope of the invention, which is defined in the claims.

### EXAMPLES

### Example 1: Encapsulation of Single Cells in Semi-Permeable Capsules

To produce gel microcapsules composed of proteinaceous shell we exemplify the use of gelatin derivative, a thermo-responsive protein, that solidifies at lower temperatures. The rheological properties of the gelatin-based gels can be controlled by the degree of substitution, polymer concentration, initiator concentration, and UV irradiation conditions. Note, that other proteins and oligopeptides, including but not limited to collagen, elastin, fibrin and silk fibroin may be similarly used.

We first generated water-in-oil droplets on a 40-μm deep co-flow microfluidics device using 3% (w/v) gelatin methacrylate (GMA) and 15% (w/v) dextran (MW˜500 k) solutions (FIG. 8a). In the experiments where the cell encapsulation was performed, the cells were suspended in dextran solution accordingly. Typical flow-rates used were: for gelatin methacrylate solution—250 μl/h; for dextran solution (with or without cells)—100 μl/h and for the carrier oil—700 μl/h. After emulsification step, the resulting emulsion droplets contained a well-centered liquid core enriched in dextran and a liquid shell enriched in GMA (FIG. 8b). The droplets were subjected to two-step polymerization process (FIG. 8c). At first, the droplets were incubated at selected temperature that induce the sol-gel transition and solidification of the gelatin shell. Next, the resulting solidified capsules were recovered from the emulsion by breaking the emulsion, re-suspended in an aqueous buffer containing photo-initiator and photo-illuminated to induce chemical cross-linking of methacrylate. More specifically, after producing water-in-oil droplets, the emulsion was transferred onto ice (˜4° C.) and incubated for ˜30 minutes to induce temperature responsive physical gelation of GMA phase. Continuing procedure on ice, capsules were recovered from the emulsion using commercial emulsion breaker (Droplet Genomics, DG-EB-1) and released into 1×PBS buffer supplemented with 0.1% (w/v) Pluronic F-68. Capsule suspension was transferred by pipetting into a new 1.5 ml tube, supplemented with 0.1% (w/v) lithium phenyl-2,4,6-trimethylbenzoylphosphinate (LAP) (Sigma-Aldrich, 900889-1G) and photo-polymerized under exposure to 405 nm light emitting diode (LED) device (Droplet Genomics, DG-BR-405) for 20 seconds. Following the aforementioned two-step polymerization procedure, we could reproducibly generate the capsules with a clear, well-centered core enriched in liquid dextran, and a solidified gel shell composed of covalently cross-linked gelatin (FIG. 8c).

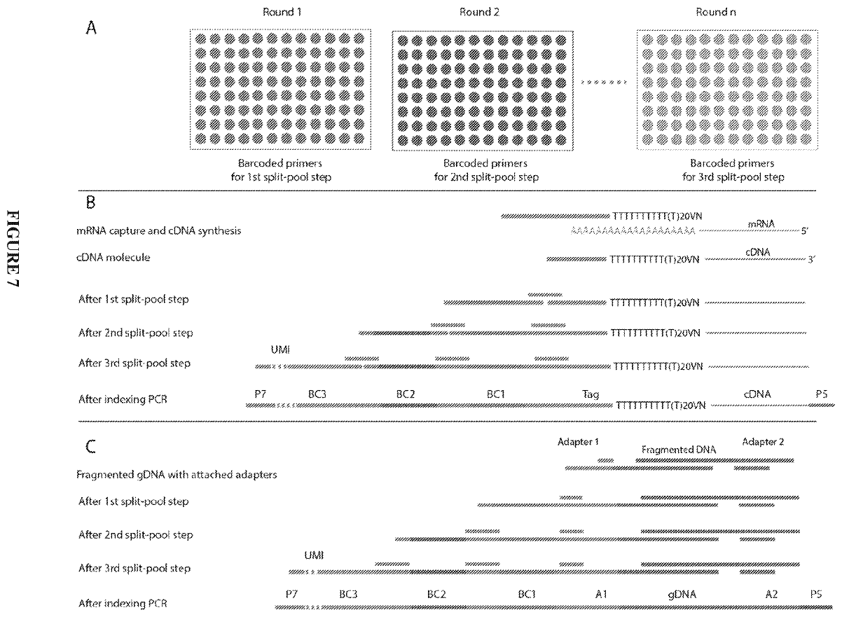

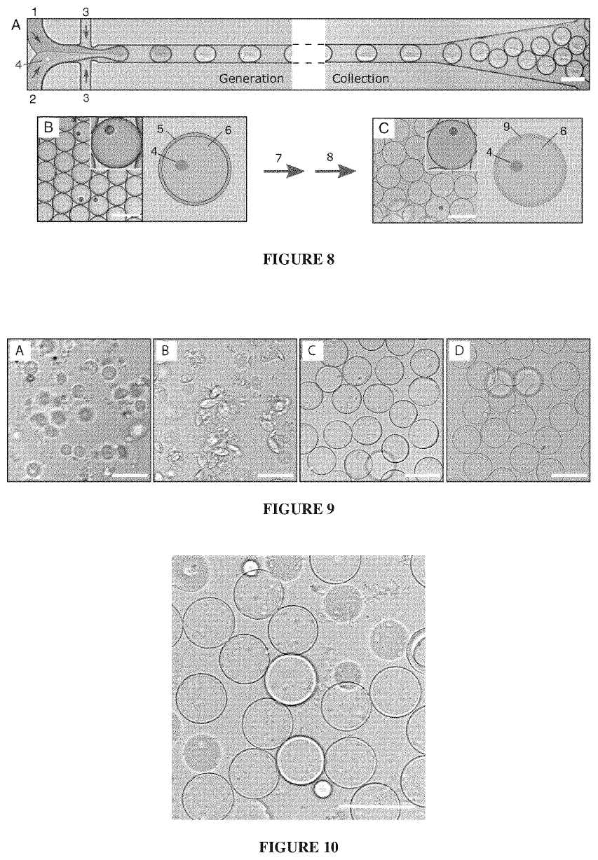

Gelatin methacrylate (GMA) will form a gel of different Young modules depending on the degree of substitution. We investigated what degree of substitution in GMA is required to achieve stable and concentric capsules. We tested capsule production using GMA with 0, 40, 60 and 80% degrees of substitution. For each test 3% (w/v) GMA with a given degree of substitution, and 15% (w/v) dextran (MW˜500 k) was used. Following the aforementioned procedure, we generated capsules and evaluated their quality under bright field microscope. Results presented in FIG. 9 show that GMA with 60 and 80% substitution ensured stable capsule generation, while GMA with 0-40% substitution failed to generate stable capsules. Increasing the GMA amount from 3% to 5% (w/v), while keeping dextran at 15% (w/v), the capsules could be generated with GMA having 40% degree of substitution (FIG. 10). Therefore, production of stable capsules depends on both the GMA substitution degree, which preferably should be at or above 40% and on the total amount of GMA used in the mix, which preferably should be at or above 3% (w/v). Stable capsule production also depends on GMA concentration, temperature, aqueous buffers.

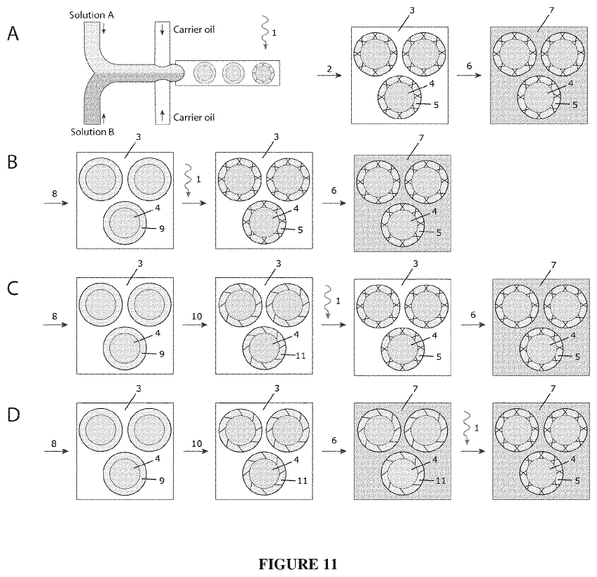

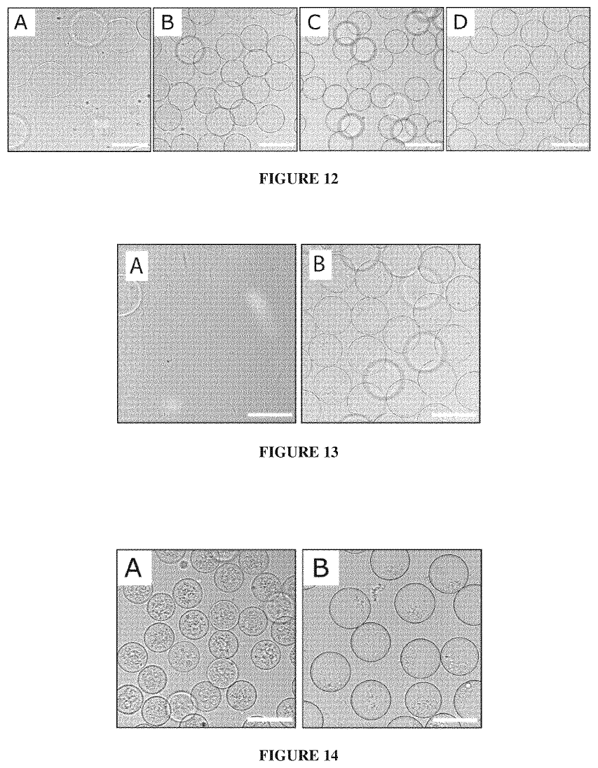

Indeed, it should be understood that there are multiple paths for producing capsules composed of proteinaceous shell and liquid core as well as capsules composed of proteinaceous shell and semi-liquid core. Some of these approaches, but not limited to, have been verified experimentally and are schematically shown in FIG. 11. In a typical scenario, the water-in-oil droplets are incubated at selected temperature that is needed to induce sol-gel transition of the shell phase (e.g. at 4° C. for 30 min). The resulting solidified capsules are then released into aqueous buffer (preferably at temperature that is below the shell melting temperature) for a desired period of time followed by chemical cross-linking of the gel shell. Different variations of this methodology are possible and some are described below:
In one example (FIG. 11A), the shell of capsules are cross-linked during droplet generation step, but only if liquid shell and liquid core has phase separated, by exposing droplets to photo-illumination. The phase separation may be a fast process and happen within a minute, or longer time scales, after water-in-oil droplet is generated. Next, the capsules are dispersed in aqueous buffer (e.g. 1×PBS buffer). The resulting capsules imaged under bright field microscope are shown in FIG. 12A.
In another example (FIG. 11B), the shell of capsules are covalently cross-linked after the emulsion collection off-chip, by photo-illuminating the collected water-in-droplets. Once polymerized, the capsules are dispersed in aqueous buffer and imaged under bright field microscope as shown in FIG. 12B.
In yet another example (FIG. 11C), the shell of capsules are polymerized in a two-step process: following emulsion collection off-chip, the liquid shell of droplets are solidified by incubating droplets at a temperature below sol-gel transition point (e.g. 4° C.) and then further cross-linked by photo-polymerization. Next, the capsules having a covalently cross-linked shell are dispersed in aqueous buffer and imaged under bright field microscope as shown in FIG. 12C.
In yet another example (FIG. 11D), the shell of capsules are polymerized in a two-step process where at first the emulsion droplets are incubated at a temperature below sol-gel transition point (e.g. 4° C.) to induce gelation of the shell, then resulting capsules are dispersed in aqueous phase and only then the shell is cross-linked by photo-polymerization. The resulting capsules are shown in FIG. 12D.

It should be understood that gel capsules having solidified shell and liquid or semi-liquid core can be generated using different means of polymerization. In a separate example, the shell of capsules can be cross-linked using chemical agent(s). The capsules shown in FIG. 13A were generated by supplementing the GMA phase with 0.3% (w/v) Ammonium Persulfate (APS), while carrier oil was supplemented with 0.4% (w/v) Tetramethylethylenediamine (TEMED). The emulsion was collected off-chip and incubated at room temperature for 2 hours to allow sufficient period of time for shell polymerization to occur. The resulting capsules were suspended in aqueous buffer and evaluated microscopically as shown in FIG. 13A.

In yet another example (FIG. 13B), the shell of capsules are polymerized using a combination of physical and chemical means. The water-in-oil droplets composed of GMA/dextran phases are collected off-chip and incubated at 4° C. for 30 min to induce physical gelation of the shell. The solidified capsules were then resuspended in aqueous buffer (lx DPBS, 0.1% (w/v) F-68) containing 0.3% (w/v) APS, 0.4% (w/v) TEMED, and incubated at room temperature for 2 h to induce chemical cross-linking of capsules' shell. The resulting capsules are shown in FIG. 13B.

It should be understood that the core of capsules is not constrained to the use of dextran. Other polyhydroxy compounds (e.g. carbohydrates, natural and synthetic polymers rich in hydroxy groups, sugars, oligosaccharides, polysaccharides) can be used instead of dextran, or different ratios of polyhydroxy compounds can be mixed at different ratios.

For example, FIG. 14 shows capsules where the dextran phase was entirely replaced with hydroxyethyl-cellulose (FIG. 14A) or Ficoll PM400 (FIG. 14B). In one example generation of capsules is achieved by replacing the dextran phase with 30% (w/v) Ficoll PM400 (Sigma-Aldrich, GE17-0300-10) solution in 1×PBS buffer. In a second example, generation of capsules is achieved by replacing the dextran phase with 3% (w/v) hydroxyethyl-cellulose (Sigma-Aldrich, 09368-100G) solution in 1×PBS buffer. In both examples the aqueous phase forming the shell contained 3% (w/v) GMA in 1×PBS buffer. Also, in both examples the capsules were generated using a microfluidics chip having 40 μm deep channels and flow rates for GMA solution were 250 μl/h, for Ficoll PM400 or hydroxyethyl-cellulose—100 μl/h and for carrier oil—700 μl/h. Emulsifications were performed at room temperature and resulting droplets were collected off-chip into 1.5 ml tube. The collected droplets were incubated at 4° C. for 30 minutes to induce gelatin solidification and thereby the capsule's shell formation. The capsules were then resuspended in an ice-cold 1×PBS buffer supplemented with 0.1% Pluronic F-68 and 0.1% (w/v) LAP and photo-polymerized under exposure to 405 nm LED device for 20 seconds. The resulting capsules were inspected under the bright field microscope and are shown in FIG. 14A and FIG. 14B.

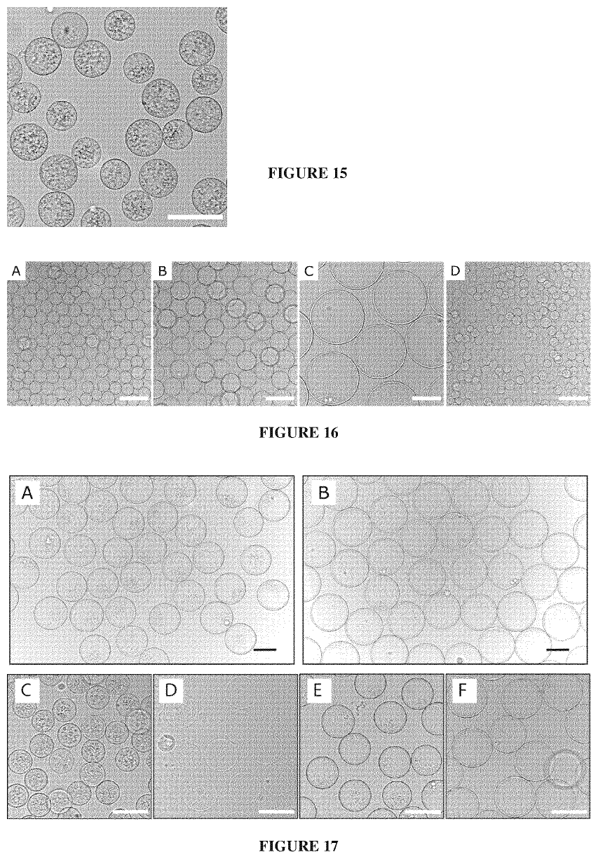

Having shown capsule production using different polyhydroxy compounds, in the following we reveal that capsules can be also generated where the core of the capsules is composed of an antichaotropic agent, such as a kosmotropic salt. To demonstrate such a possibility, we generated capsules where the core forming phase was ionic liquid based on ammonium sulfate. Capsules were generated using a microfluidics chip having 40 μm deep channels and the flow rate for 3% (w/v) GMA solution at 175 μl/h, for 1M ammonium sulfate at 175 μl/h and for carrier oil at 700 μl/h. Emulsification was performed at room temperature and resulting droplets were collected off-chip into 1.5 ml tube. The collected droplets were then incubated at 4° C. for 30 minutes to induce gelatin solidification and thereby the capsule's shell formation. Continuing procedures on ice, the capsules were recovered from the emulsion and resuspended in an ice-cold 1×PBS buffer supplemented with 0.1% (w/v) Pluronic F-68 and 0.1% (w/v) LAP and photo-polymerized under exposure to 405 nm LED device (Droplet Genomics, DG-BR-405) for 20 seconds. The resulting capsules were inspected under the bright field microscope and are shown in FIG. 15.

Although in the examples above preferably only the shell of capsules is solidified while keeping the core of the capsules in a liquid form, it should be understood for the experienced in the field that capsule core can be also polymerized into a desirable strength gel mesh by adding a cross-linking agent soluble in the core phase (or alternatively soluble in both core/shell forming phases). In such case the capsules will contain solidified shell and solidified core of different stiffness. One such example has been revealed by our previous invention (US20200400538A1), where the cross-linking agent, PEGDA, distributed in both phases yet with a higher fraction at the shell phase.

When using a microfluidics device having channels of different depth ranging from 20 to 80 μm deep, the size of capsules could be tuned between 35 and 200 μm by simply changing the flow rates of the system, without significantly affecting their concentricity, thus offering flexibility in size for diverse assay. More specifically, using microfluidics device 20 μm deep the size of capsules was in the range of 35 to 45 μm (FIG. 16A). Using microfluidics device 40 μm deep the size of capsules could be tuned between 60 and 85 μm (FIG. 16B). Using microfluidics device 80 μm deep the size of capsules was in the range of 150 to 200 μm in diameter (FIG. 16C). It should be possible to generate even smaller or larger capsules by employing a microfluidic system having a smaller or larger cross-section channels, or having a smaller/larger size nozzle, respectively. Alternatively, the smaller size capsules can be generated by employing a geometrically mediated breakup of droplets [34]. For example, capsules shown in FIG. 16D were generated using a geometrically mediated breakup, which resulted in 24 μm diameter capsules.

As explained below capsule size can be controlled not only by the flow rates or the cross-section of the microfluidic channels, but also by the temperature. To prove the effect of temperature on capsules size the GMA/dextran capsules were generated using a microfluidics chip having 80 μm deep channels. The flow rates for GMA solution were 200 μl/h, for dextran—50 μl/h and for carrier oil—500 μl/h. Emulsion was collected off-chip into 1.5 ml tube at room temperature and placed at 4° C. for 30 minutes to induce gelatin solidification. Next, emulsion was divided into two fractions and processed separately at different temperatures.
The capsules in the first fraction were recovered from the emulsion, resuspended in an ice-cold 1×PBS buffer supplemented with 0.1% (w/v) Pluronic F-68 and 0.1% (w/v) LAP, and photo-polymerized under 405 nm wavelength for 20 seconds. The resulting capsules are shown in FIG. 17A.
The capsules in the second fraction were recovered from the emulsion, resuspended in an ice-cold 1×PBS buffer supplemented with 0.1% (w/v) Pluronic F-68 and 0.1% (w/v) LAP, and incubated at room temperature (˜22° C.) for 15 min to allow capsule swelling to occur. Following incubation, the capsules were photo-polymerized under 405 nm wavelength for 20 seconds. The resulting capsules are shown in FIG. 17B.

In yet another example the effect of temperature on capsule size is revealed by producing capsules with a core composed of polyhydroxy compounds other than dextran. The capsules of choice were composed of GMA/hydroxyethyl-cellulose or GMA/Ficoll PM400 blend. In one example, the first aqueous phase contained 30% (w/v) Ficoll PM400 solution in 1×PBS buffer and the second aqueous phase contained 3% (w/v) GMA in 1×PBS. In another example, the first aqueous phase contained 3% (w/v) hydroxyethyl-cellulose solution in 1×PBS and the second aqueous phase contained 3% (w/v) GMA in 1×PBS buffer. In both examples the capsules were generated using a microfluidics chip having 40 μm deep channels and flow rates for GMA phase at 250 μl/h, for Ficoll PM400 or hydroxyethyl-cellulose—100 μl/h and for carrier oil—700 μl/h. Emulsifications were performed at room temperature. The collected droplets were incubated at 4° C. for 30 minutes to induce gelatin solidification and thereby the capsule's shell formation. Next, emulsion was divided into two fractions. The emulsion in the first fraction was further processed on ice, whereas the emulsion in the second fraction was processed at room temperature following incubation on ice.
The capsules in the first fraction were dispersed in an ice-cold 1×PBS buffer supplemented with 0.1% (w/v) Pluronic F-68 and 0.1% LAP, and photo-polymerized under 405 nm light for 20 seconds. The resulting polymerized capsules were inspected under the bright field microscope and are shown in FIG. 17C and FIG. 17E.
The capsules in the second fraction were resuspended in an ice-cold 1×PBS buffer supplemented with 0.1% (w/v) Pluronic F-68 and 0.1% LAP, and transferred to room temperature (˜22° C.) for 15 min to induce the capsule swelling and expansion. Following the incubation at room temperature the capsules were photo-polymerized under 405 nm light for 20 seconds. The resulting capsules are shown in FIG. 17D and FIG. 17F.
   In summary, the results presented in FIG. 17 prove that capsule size can be controlled by temperature, and more specifically that preincubation of capsules at temperature higher than 4° C., prior to the cross-linking step, leads to larger size capsules.

In addition to achieving the precise control over the capsule size, the shell thickness could be also tuned by adjusting the flow rates of the system or the concentration of the shell forming polymer. In one example, capsules generated using a microfluidics device 20 μm deep had a shell 2 μm thick (FIG. 16A). In another example, capsules generated using a microfluidics device 40 μm deep had a shell 3 μm thick (FIG. 16B). In yet another example, capsules generated using a microfluidics device 80 μm deep had a shell 5 μm thick (FIG. 16C).

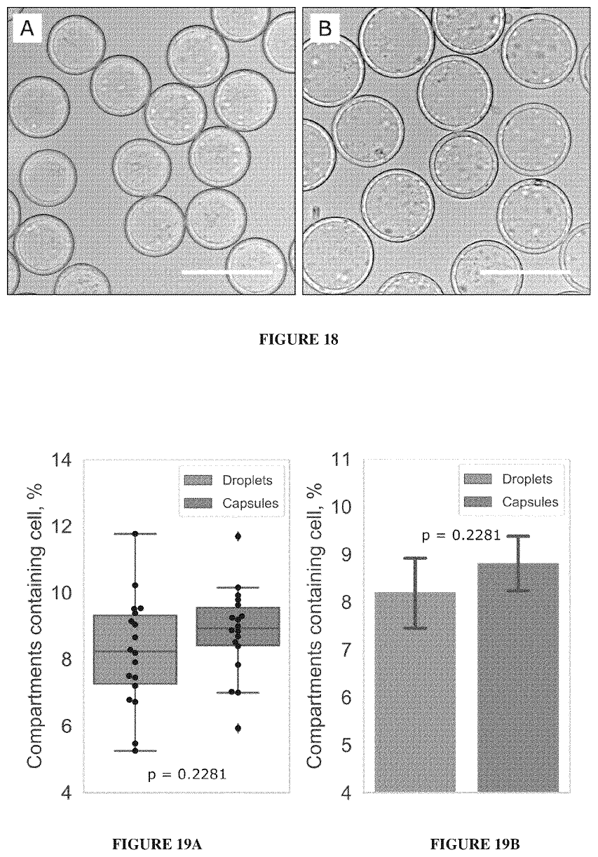

The shell thickness of the capsules could be also tuned by adjusting the concentration of the shell forming polymer. FIG. 18A shows 68 μm size capsules having 6.5 μm shell and 55 μm core. Such capsules can be generated by emulsifying 5% GMA with 15% dextran solutions followed by physical gelation and chemical cross-linking of the shell. As expected, preincubation at room temperature (˜22° C.) for 15 min before photo-polymerization increased the size of capsules to ˜82 μm diameter (70 μm core and 6 μm shell) as shown in FIG. 18B.

Altogether the examples presented above prove that capsule generation is highly flexible and is not restricted to one type of material. Capsule shell can be hardened using different agents such as temperature, light or chemicals. The capsule size and shell thickness can be tuned by changing the volumetric ratios of fluids during emulsification, the concentration of the ingredients in the liquid phases, the share force generated by carrier oil, or by changing the temperature at which capsules are generated/or processed. It should be understood that these examples are not limited.

To evaluate cell encapsulation and retention efficiency we used K-562 (ATCC, CCL-243) and HEK293 (ATCC, CRL-1573) cell lines. We quantified cell retention microscopically immediately after encapsulation, and then after capsule formation, and confirmed that the gel capsules efficiently retained encapsulated cells (FIG. 19). No significant difference was detected (two-tailored t-test (n=18) t=1.22, p=0.2281) confirming that compartmentalized cells were efficiently retained through all the steps of capsule generation. As revealed in the example below the gel capsules based on proteinaceous shell were compatible with multi-step biochemical and biological reactions; enabled passive exchange of low molecular weight compounds; retained large molecular weight compounds such as RNA or DNA inside; and the capsules were prone to user-controlled disintegration upon enzymatic treatment for releasing the internal capsule's content (e.g. encapsulated cell, PCR amplicon).

### Example 2: Performing Single-Cell RT-PCR Using Semi-Permeable Capsules

To identify the individual cells based on their gene expression profile, we performed a proof-of-concept study using a mixture of K562 and HEK293 cells. The cell lines were mixed at ratio 1:1 and encapsulated at a limiting dilution such that each capsule, on average, would contain no more than one cell. Cell encapsulation using droplet microfluidics is a well described process governed by Poisson statistics. As a reference, we also separately encapsulated either K562 or HEK293 cells. After cell encapsulation, the water-in-oil droplets were converted to gel capsules using a two-step polymerization approach as detailed above. At first the emulsion droplets were incubated at 4° C. temperature to induce gelation of the shell, and then resulting capsules were dispersed in aqueous phase and cross-linked by photo-polymerization. Next, the capsules were suspended in GeneJET RNA Purification Kit Lysis Buffer (Thermo Scientific, K0732) containing 40 mM DTT and centrifuged immediately. The supernatant was aspirated and replaced with 1 mL of fresh lysis buffer followed by incubation at room temperature (21° C.) for 5 minutes. Following the incubation, the capsules were rinsed once in 1 mL lysis buffer and five-times in 1 mL washing buffer (10 mM Tris-HCl [pH 7.5] containing 0.1% (v/v) Triton X-100). During the washing steps the centrifugation was performed at 2000 g for 2 min.

Next, the genomic-DNA was depleted by adding 100 μl of close-packaged capsules to 200 μl reaction mix containing 0.05 U/μl DNAse I enzyme (Thermo Scientific, K2981) and 0.2 U/μl RNase Inhibitor (Thermo Scientific, EO0381) followed by incubation at 37° C. for 20 minutes. Next, 5 units of DNase I enzyme were added in a reaction mix and incubated at 37° C. for 10 minutes. After DNAse I treatment, the capsules were rinsed three-times in washing buffer (10 mM Tris-HCl [pH 7.5] containing 0.1% (v/v) Triton X-100) and subjected to a reverse transcription (RT) reaction.

The cDNA synthesis was performed in 200 μl reaction mix, containing 100 μl close-packaged capsules, 1×RT Buffer (Thermo Scientific, EP0751), 1× Oligo(dT)18 Primer 5 (Thermo Scientific, SO131), 0.5 mM dNTP Mix (Thermo Scientific, R0192), 5 U/μl Maxima H Minus Reverse Transcriptase (Thermo Scientific, EP0751), 0.2 U/μl RiboLock RNase Inhibitor and incubated at 50° C. for 60 minutes. After cDNA synthesis, the capsules were rinsed three-times in washing buffer (10 mM Tris-HCl [pH 7.5] containing 0.1% (v/v) Triton X-100) and then subjected to polymerase chain reaction (PCR). The PCR was performed in 100 μl reaction volume by mixing 47 μl of closely-packed capsules with 53 μl of PCR reaction mix (Table 1).

TABLE 1
PCR mix composition

| Volume | Material | Final |
|---|---|---|
| 50 μl | 2x Phire Tissue Direst PCR Master | 1x |
| | Mix | |
| 47 μl | Close-packed capsules | — |
| 0.5 μl | 100 μM 555-YAP-forward | 0.5 μM |
| 0.5 μl | 100 μM YAP-reverse | 0.5 μM |
| 0.5 μl | 100 μM 488- PTPRC -forward | 0.5 μM |
| 0.5 μl | 100 μM PTPRC -forward | 0.5 μM |
| 0.5 μl | 100 μM 647-ACTB-forward | 0.5 μM |
| 0.5 μl | 100 μM ACTB-forward | 0.5 μM |
| 100 μL | Final | — |

During the PCR, the specific markers preferentially expressed in HEK293, K562 or in both cell lines, were amplified. Specifically, the cDNA of YAP, PTPRC and ACTB markers, was amplified using marker specific primer set listed in Table 2. The primer set consisted of three primer pairs targeting the cDNA of YAP, PTPRC and ACTB transcripts. Indeed, it should be understood that other markers can be targeted during the PCR step.

TABLE 2
The list of multiplex PCR primers used to
amplify the markers of interest

| | | | Product |
|---|---|---|---|
| | Name | Sequence (5′-3′) | length |
| SEQ ID 1 | 555-YAP- | Alexa Fluor 555-CC | 550 bp |
| | forward | CTCGTTTTGCCATGAACC | |
| SEQ ID 2 | YAP- | CCAGTGTTCCAAGGTCC | |
| | reverse | ACA | |
| SEQ ID 3 | 488-PTPRC- | Alexa Fluor 488-ACA | |
| | forward | TTGCTGCACAAGGTCCCAG | |
| SEQ ID 4 | PTPRC- | AACCATCAGGCATCTCTGT | |
| | reverse | CGC | |
| SEQ ID 5 | 647-ACTB- | Alexa Fluor 647-AT | 599 bp |
| | forward | TCCTATGTGGGCGACGA | |
| SEQ ID 6 | ACTB- | AATGGTGATGACCTGGCCG | |
| | reverse | | |

Each primer pair contained a sequence specific oligonucleotide, fluorescently labelled at 5′ end, that served as a forward primer. The reverse primer was not labelled with the fluorescent dye. Oligonucleotides targeting different markers carried different fluorescent dyes emitting light at different wavelength thus enabling differentiation of gene expression based on the fluorescence signal. During the PCR, the fluorescently labelled oligonucleotides were incorporated into the PCR amplicons, turning an amplified DNA into a fluorescent product. Although, as exemplified here, only forward primer contained a fluorescent dye, it should be understood to the expert in the field that it should be possible to use both reverse and forward primers labelled with a fluorescent dye and by doing so increase the fluorescence signal intensity of PCR amplicon. Target amplification was performed for 30 cycles with Phire Tissue Direct PCR Master Mix (Thermo Scientific, F170L) using thermocycling conditions provided in Table 3.

TABLE 3
The PCR thermocycling conditions

| Step | Temp | Time | | No Cycles |
|---|---|---|---|---|
| Initial | 98° C. | 5 | min | |
| denaturation | | | | |
| Amplification | 98° C. | 5 | s | 30 |
| | 64° C. | 5 | s | |
| | 72° C. | 20 | s | |
| Final extension | 72° C. | 1 | min | 1 |

After the PCR, 100 units of Exonuclease I (NEB, M0293L) enzyme was added directly to post-PCR mix, incubated at 37° C. for 15 minutes and rinsed three-times in washing buffer (10 mM Tris-HCl [pH 7.5] containing 0.1% (v/v) Triton X-100) to remove the excess of fluorescently labeled forward primers that have not been incorporated into PCR amplicons. The capsules then were analyzed using a fluorescence microscopy and flow cytometry.

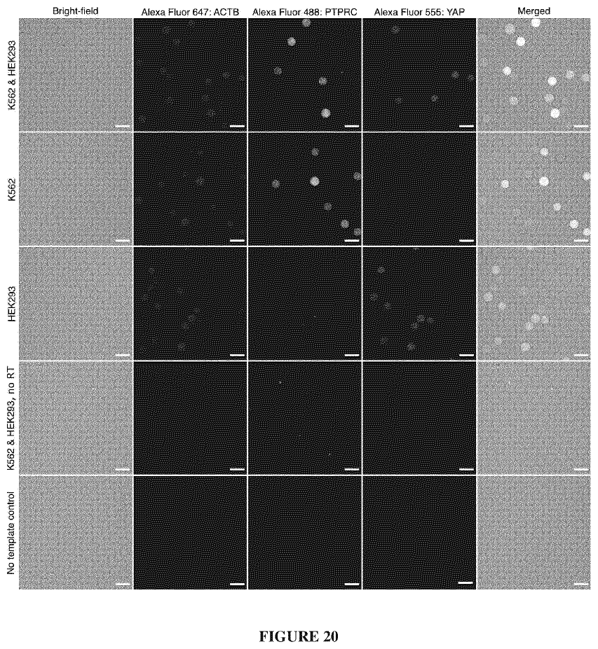

Given the differential expression of PTPCR and YAP markers in K562 and HEK293 cells, the capsules harboring K562 cell should be positive in PTPCR marker, while capsules harboring HEK293 cell should be YAP positive. In addition, both capsule types should be positive in ACTB marker since this gene is ubiquitously expressed in both cell types. Indeed, fluorescence microscopy analysis confirmed that capsules harboring either K562 cells or HEK293 cells alone were distinguishable by expression of PTPRC or YAP gene marker, respectively (FIG. 20) and that both cells expressed ACTB. When capsules containing a mixture of cells were inspected under the fluorescence microscope, two distinct populations corresponding to K562 and HEK293 cells were detected based on either PTPRC-ACTB or YAP-ACTB positive counts (FIG. 20). Interestingly, a third population showing a fluorescent signal corresponding to ACTB target (FIG. 20) was also detected, indicating that capsule-based multiplex RT-PCR assay can detect the ambient RNA molecules originating from the lysed cells. Performing the same assay on empty capsules (no template control) as well as on capsules without reverse transcription enzyme but having K562 and HEK293 cells (no RT control), the ACTB positive capsules were not detected (FIG. 20).

### Example 3: Split-Pool Method for Barcoding Nucleic Acids

Protocols for the performance of the split-pool method are provided below.

### Single-Cell Transcriptomics (Related to FIG. 3)

First, the plurality of microcapsules comprising plurality of single-cells are generated. The cells of interest (e.g. K-562 cells) may be re-suspended in a solution having polyhydroxy compound (e.g., 15% dextran solution (MW 500 k) (Sigma-Aldrich, 31392-10G)) at a desirable concentration so that during encapsulated step the microcapsule contains, on average, no more than 1 cell. Cells are introduced into a microfluidics device along with polyampholyte solution (e.g., gelatin methacrylate (Sigma-Aldrich, 900496-1G)) and the carrier oil (e.g., HFE-7500 oil supplemented with 2% fluorosurfactant). The water-in-oils droplets comprising cells, polyampholyte and polyhydroxy compound are generated using a microfluidics chip. Encapsulations are preferentially performed at room temperature (˜23° C.). The resulting water-in-oil droplets are collected off-chip into a laboratory tube, which may be prefilled with 200 μl of light mineral oil (Sigma-Aldrich, M5904-500ML). After encapsulation, the emulsion is transferred to 4° C. and incubated for 30-60 minutes to induce solidification of the microcapsule's shell. The resulting intermediate-microcapsules, having a solidified polyampholyte shell, are recovered from the emulsion using commercial emulsion breaker (Droplet Genomics, DG-EB-1) and resuspended in ice-cold 1×PBS, supplemented with 0.1% Pluronic F-68 (Gibco, 24040032). The intermediate-microcapsule suspension is transferred into a new laboratory tube, supplemented with 0.1% (w/v) lithium phenyl-2,4,6-trimethylbenzoylphosphinate (Sigma-Aldrich, 900889-1G) and cross-linked (photo-polymerized) under exposure to 405 nm LED device (Droplet Genomics, DG-BR-405) for 20 seconds. Alternatively, the intermediate-microcapsule suspension may be treated with transglutaminase enzyme (e.g., available at Sigma, cat no. SAE0159-25UN) to induce the cross-linking of the microcapsule's shell. After cross-linking reaction, the microcapsules are rinsed twice in 1×PBS buffer containing 0.1% Pluronic F-68, and then resuspended in a desirable buffer (e.g. lysis buffer, neutral buffer, etc.).

### Cell Lysis.

The microcapsules are then treated with lysis buffer in order to lyse the cells and release the cellular RNA molecules. Cell lysis reagents may be chosen from a variety of detergents available commercially (see for example, Srirama M. Bhairi, Chandra Mohan, Sibel Ibryamova, Travis LaFavor, Detergents: A guide to the properties and uses of detergents in biological systems) and person with experience in the art will be able to choose appropriate lysis conditions. For example, lysis buffer may include non-ionic detergents such as NP40, Igepal series, Triton series, or other. Lysis buffer may include ionic detergents such as SDS. It may include chaotropic agents, protein-denaturating agents such as guanidinium. In some preferred cases, the lysis reagent is NP40 detergent. In some other preferred cases, the lysis buffer is composed of 4M Guanidinium thiocyanate, 55 mM Tris-HCl (pH 7.5), 25 mm EDTA, 3% (v/v) Triton X-100. In some scenarios the microcapsules may be treated with DNAse enzyme to digest genomic DNA of encapsulated (and lysed) cells.

### Wash Microcapsules.

After the lysis step the cell lysate may be washes in a washing buffer several times until most or all lysis reagents are removed. During the lysis and washing lysis steps the centrifugation of the microcapsule suspension can be between 100-20.000 g. Washed capsules can be transferred into a separate tube to perform next round of reactions.

### Performing Reverse Transcription on Encapsulated Lysed-Cells

To initiate the copy DNA (cDNA) synthesis on nucleic acids of lysed cells, the capsules comprising lysed cells may be resuspended in a washing buffer supplemented with RT primer having a desirable ligation adapter at 5′ end. (5′-[ligation adapter]-TTTTTTTTTTTTTTTTTTTTTTVN 3′ (SEQ ID 7). In some limited scenarios the ligation adapter may comprise a barcode sequence, where the barcode is a nucleotide sequence preferably 6-8 nt long. RT primer is annealed to mRNA and RT reaction is initiated by loading microcapsules into RT reaction mix comprising RT enzyme. For example, in a specific embodiment the RT reaction mix may contain: 175 μL of RT Master Mix contains 100 μL of 5×RT Buffer, 6.25 μL of 40 U/μL RiboLock Rnase Inhibitor, 25 μL of 200 U/μL Maxima H Minus Reverse Transcriptase, and 43.75 μL of nuclease-free water and 325 μL of capsules suspension. In some scenarios, the RT reaction mix may comprise TSO primer (5′-AAGCAGTGGTATCAACGCAGAGTACATrGrGrG) (SEQ ID 8). The reverse transcription (RT) reaction is performed at a desirable temperature (e.g. 50° C.) and terminated at 85° C. for 5 min.

Performing Rounds of Split-Pool on Microcapsules Carrying cDNA (See Also to FIG. 7-B)
1) The plurality of microcapsules carrying cDNA derived from plurality of individual cells is distributed equally into N₁ wells, where each well carries one of N₁ distinct DNA primers (barcoded primers). The said DNA primers, here is referred as simply barcodes, might be ssDNA, dsDNA or a combination of ssDNA and dsDNA.
2) The barcodes are ligated to cDNA (preferably 5′end of cDNA).
3) After ligation the unbounded primers are washed away and microcapsules pooled together.
4) At this step, the number of unique combinations of barcodes is N₁
5) The microcapsules now having barcodes N₁ are split equally into N₂ pools (typically N₁=N₂), and each pool is distributed equally into N₂ wells, where each well carries one of N₂ distinct DNA primers (barcodes).
6) After ligation and pooling, the number of unique combinations of barcodes will increase up to N₁×N₂
7) Steps 1-6 may be repeated one or few times to increase the unique combinations of barcodes (e.g., N₁×N₂×N₃).
8) After the split-pool indexing the microcapsules shall contain the barcoded cDNA derived from single-cells, and where the barcode combination (nucleotide sequence of barcoded primers) in one microcapsule is different from the barcode combinations in other microcapsules.
9) During the final round of split-pool the DNA primers may include so called unique molecular identifier (UMI), which comprised 6-12 random nucleotide sequence. The UMIs are used to correct PCR amplification biases that occur during library preparation.
10) The ligation reaction may be halted by addition of inhibitors such as EDTA, vanadium, heating, or by other means.
11) The microcapsules are pooled together and washed to remove the enzymes, excess of unligated molecules, side products, etc.
12) The cDNA having ligated DNA fragments may be converted into single stranded cDNA by removing the DNA molecules through denaturation, for example by washing repeatedly in 0.1M sodium hydroxide, or by other alternative means.
13) The barcoded-cDNA may be released from microcapsules by dissolving the capsules.
14) The barcoded-cDNA may be amplified by PCR.
15) Amplified cDNA may be submitted to DNA sequencing.
16) Before sequencing it may be preferential to fragment the amplified cDNA (see below for details) into smaller fragments (approximately 500 bp long), purified, ligated to PCR adapters, and re-amplified by PCR.
17) After sequencing, each transcriptome is assembled by combining the reads containing the same barcode combinations.

### Fragmentation of the Barcoded-cDNA Library:

To perform fragmentation of the amplified cDNA library one can use enzymatic, physical or chemical means. In some scenarios it may be preferable to use enzymatic fragmentation (e.g., NEBNext Ultra DNA Library Prep Kit for Ilumina, cat no. #E7645) or a tagmentation reaction (e.g., Nextera XT DNA library preparation kit). After fragmentation the library is ligated to PCR adapter and may be amplified by PCR (preferably 9 to 12 rounds of PCR). The resulting library is purified using magnetic particles (e.g., SPRI beads).

### Sequencing:

Following the instruction of the DNA sequencing provider (e.g., Illumina) for sequencing the DNA libraries. When using Illumina sequencing instrument, it may be preferred to sequence the transcriptome using following cycles: Read 1—≥70 cycles, Read 2—6-8 cycles, Read 3—≥100 cycles.

### Single-Cell Genomics (Related to FIG. 6)

For single-cell genome sequencing applications, first the plurality of microcapsules comprising plurality of cells are generated as described above. Briefly, cells of interest are encapsulated into microcapsules and are lysed to release the cellular nucleic acids. If desirable the mRNA may be digested with RNAse enzyme. The cell nucleus may be disrupted to release the genomic DNA. For single-cell genomics applications it may be desirable to use lysis conditions that break chromatin structure and/or disrupt cell nucleus. For that purpose, the use of heating, guanidinium salts, ionic detergents, may be advantageous. Following cell lysis, the microcapsules may be washed in a desirable buffer to remove the lysis reagents, yet retain the nucleic acids. Next, the microcapsules, having lysed cells, may be dispersed in suitable reaction conditions to initiate the whole genome amplification (WGA) reaction. The WGA can be initiated by DNA replication enzyme such as phi29 DNA polymerase, Bst DNA polymerase, etc. After WGA, the resulting DNA may be subjected to a fragmentation reaction. Indeed, the fragmentation of the genomic DNA may be conducted prior performing WGA. To perform the DNA fragmentation the microcapsules may be resuspended in a reaction buffer containing the transposase enzyme. In some embodiments the microcapsules may be resuspended in a reaction buffer containing endonucleases (e.g., restriction endonuclease). In some embodiments the microcapsules may be resuspended in a reaction buffer containing nuclease (e.g., DNAse I). The genomic DNA may be fragmented chemically (e.g., using complexes that generate hydroxyl radicals such as iron-EDTA complex). The genomic DNA may be fragmented physically (e.g., using ultrasound). Upon chemical, enzymatic or physical fragmentation the resulting DNA fragments may be longer than 1 base pair (bp) and shorter than 1000.000 bp, and more preferably large than 100 bp and smaller than 10 kbp, and even more preferably majority of fragments being in the range of 200-2000 bp. In some embodiments the microcapsules comprising fragmented DNA may be resuspended in a reaction buffer containing ligase and suitable DNA adapters. The DNA adapters may be ligated to fragmented DNA by ligase catalyzed reaction. Once ligated to DNA adapters the barcoding reaction may be conducted by performing split-and-pool (combinatorial indexing) reaction.

Performing dsDNA Barcoding by Split-Pool (See Also to FIG. 7C)
1) The plurality of microcapsules carrying DNA derived from individual cells, are distributed equally into N₁ wells, where each well carries one of N₁ distinct DNA adapters (barcodes).
2) The barcodes from 1^{st} pool are ligated to dsDNA.
3) After first ligation, the barcodes are washed away and the microcapsules are pooled.
4) The microcapsules are then split equally into N₂ pools (typically N₁=N₂), and each pool is distributed equally into N₂ wells, where each well carries one of N₂ distinct DNA adapters (barcodes).
5) After second ligation reaction, the 2^{nd} pool of barcodes are ligated to DNA.
6) The microcapsules may be washed to remove the unligated primers, and the microcapsules are pooled again.
7) The number of unique combinations of barcodes is now N₁×N₂
8) The procedure is repeated until a desirable number of barcodes is obtained. For example, performing split-pool procedure 3-times and using 96-well plates., 96\hat{ }3 barcode combinations are obtained (e.g., 96×96×96=884736).
9) Performing split-pool procedure 3-times using 384-well plates will result over 56 million unique barcode combinations (e.g., 384\hat{ }3).
10) After the combinatorial split-pool indexing the microcapsules shall contain the barcoded individual genomes derived from single-cells, and where the barcode combination (nucleotide sequence of barcoded primers) in one microcapsule is different from the barcode combinations in the other microcapsules.
11) During the final round of split-pool the DNA primers may include so called unique molecular identifier (UMI), which comprised 6-12 random nucleotide sequence. The UMIs are used to correct PCR amplification biases that occur during library preparation.
12) The barcoded DNA can then be amplified inside the microcapsules, or it can be released from microcapsules and amplified in bulk.
13) Amplified DNA may be submitted to DNA sequencing. In some scenarios, amplified DNA may be further fragmented, purified or treated enzymatically before submitting the library to DNA sequencing.
14) After the sequencing, the individual genomes are assembled by combining the reads containing the same barcode combinations.

### Amplification of the Barcoded Genomic Library:

Wash the microcapsules comprising the barcoded genome in a desirable buffer (e.g. PCR buffer), transfer the microcapsules to the tube and initiate the PCR reaction. It may be preferable to use KAPA HiFi HotStart for performing the PCR reaction. To amplify the barcoded genome the PCR may comprise 4-20 cycles, preferably around 12 cycles of PCR. When performing DNA amplification in bulk, break the microcapsules by treating them with protease enzyme, collect the released barcoded-DNA and amplify by PCR.

### Sequencing:

Following the instruction of the DNA sequencing provider (e.g., Illumina) sequence the DNA libraries. When using Illumina sequencing instrument, it may be desirable to sequence the library using following parameters: Read 1—≥75 cycles, Read 2—≥90 cycles, Read 3—6-8 cycles, and Read 4—≥75 cycles.

### Single-Cell Epigenomics (Partly Related to FIG. 4)

For single-cell epigenetic profiling applications, first the plurality of microcapsules comprising plurality of cells are generated as described above. Briefly, the cells of interest are encapsulated into microcapsules and are lysed to release the nucleic acids. The cell nucleus may be kept intact, or disrupted. The experienced person in the field will be aware of the methods to lyse cells, and keep cell nucleus intact or disrupted. For example, using ionic detergent (such as SDS), the cell nucleus may be disrupted, while using non-ionic detergents e.g., Triton-X100 the disruption of cell nucleus is mild and insignificant. It is desirable to use lysis conditions that are not detrimental, or only minimally detrimental, to chromatin structure. Upon cell lysis, the cells may be washed in a desirable buffer to remove lysis reagents. The microcapsules having lysed cells may be dispersed in suitable reaction conditions to initiate so called the epigenetic profiling reaction. The goal of epigenetic profiling reaction is to fragment the open (accessible) chromatin. For example, in some embodiments the microcapsules comprising lysed cells may be resuspended in a reaction buffer containing the transposase enzyme, whereas the transposase enzyme may be assembled into a tertiary complex comprising dsDNA adapters. To load Tn5 with adapters, the dsDNA primers (e.g., annealed PCR Nextera adapters) are mixed with Tn5 enzyme and incubated at a desirable temperature (e.g., room temperature) for approximately 60 minutes to allow sufficient time for a tertiary complex formation (often called as “loaded Tn5”).

### Chromatin Fragmentation:

To fragment the genomic DNA the microcapsules may be treated with enzyme belonging to one of the types: nuclease, endonuclease or transposase. In a preferred scenario DNA fragmentation (tagmentation of the chromatin) is performed with loaded Tn5. The tagmentation reaction may be performed at a desirable temperature such as 37° C. for 60 minutes and then stopped by washing the capsules in another buffer one or several times. Next, the DNA adapters may be ligated to the tagmented DNA. Alternatively, the dsDNA adapters may be extended by the primer-extension reaction. By suspending the microcapsules in a suitable reaction buffer that includes the DNA adapters and/or primers, enzymes, RNAse inhibitor, dNTPs, the said reagents will diffuse into the core of the microcapsule and will participate in the ligation. After ligation reaction, the microcapsule shall contain the fragmented genome (tagmented DNA) attached to DNA adapters. Alternatively, the fragmented DNA can be attached to DNA adapters by performing primer extension reaction driven by reverse transcriptase, and more preferably by DNA polymerase such as Klenow, Bst DNA polymerase or other.

Once fragmented DNA is attached to the DNA adapters the microcapsules may be subjected to combinatorial indexing by split-pool as described above in “Performing dsDNA barcoding by split-pool” section. Finally, the barcoded-DNA may be released from microcapsules and processed and sequenced as described above. After the sequencing, the individual genomes are assembled by combining the reads containing the same barcode combinations.

### Single-Cell Methylomics (Related to FIG. 6)

For single-cell methylome profiling applications, first the plurality of microcapsules comprising plurality of cells are generated as described above. Following cell lysis and purification the genomic DNA is fragmented within the microcapsules by resuspending the microcapsules in a reaction buffer containing enzyme belonging to nuclease or transposase class. The genomic DNA may be also fragmented chemically (e.g., using complexes that generate hydroxyl radicals such as iron-EDTA complex), or physically (e.g., using ultrasound). Upon chemical, enzymatic or physical fragmentation the resulting DNA fragments may be retained inside the microcapsule and preferentially should be in the range of 200-2000 kb. The said DNA fragments are then ligated to DNA adapters by a ligase catalyzed reaction. Once ligated to DNA adapters the barcoding reaction may be conducted by performing split-and-pool (combinatorial indexing) reaction as described above in “Performing dsDNA barcoding by split-pool” section. The barcoded DNA fragments can then be applied to bisulfite conversion, a process in which the deamination of unmethylated cytosines into uracils occurs, while methylated cytosines (both 5-methylcytosine and 5-hydroxymethylcytosine) remain unchanged. Alternatively, the fragmented DNA can be subjected to TET/pyridine borane treatment within the microcapsules, or outside the microcapsules (after disruption of the microcapsules with protease). The TET/pyridine borane sequencing enables detection of 5-mehylcytosine (5mC) and 5-hydroxymehylcytosine (5hmC). TET catalyzed oxidation of 5mC and 5hmC to 5-carboxylcytosine (5caC), while pyridine borane reduction of 5caC to dihydrouracil (DHU) and subsequent PCR conversion of DHU to thymine, enabling a C-to-T transition of 5mC and 5hmC. The post-TET/pyridine borane treated DNA fragments can be purified, converted to DNA library by adding sequencing adapters, amplified and sequenced. After the sequencing, the individual genomes and methylation positions are assembled by combining the reads containing the same barcode combinations.

### Simultaneous Single-Cell Transcriptomics and Epigenomics (Related to FIG. 4)

### Cell Encapsulation:

For simultaneous single-cell transcriptomics and epigenomics profiling, at first the plurality of microcapsules comprising plurality of single-cells are generated as described above. Briefly, cells of interest are encapsulated into microcapsules and are lysed to release the mRNA. The cell nucleus may be kept intact, or disrupted depending on the exact biological question and application. The experienced person in the field will be aware of the methods to lyse the cells and cell nucleus. For example, using ionic detergents, e.g. SDS reagents the cell nucleus may be disrupted, while using non-ionic detergents e.g., Triton-X100 the disruption of cell nucleus is insignificant. It is desirable to use the lysis conditions that are not detrimental, or only minimally detrimental, to chromatin structure. Upon cell lysis, the microcapsules my washed in a desirable buffer to remove lysis reagents and purify cell lysate. The microcapsules having lysed cells may be dispersed in suitable reaction conditions to initiate the enzymatic reaction. For example, in some embodiments the microcapsules may be resuspended in a reaction buffer containing transposase enzyme, whereas the transposase enzyme as described above (3. Single-cell epigenomics). Follow the chromatin fragmentation procedure described above (3. Single-cell epigenomics, Chromatin fragmentation).

cDNA Synthesis:

To convert RNA to cDNA the microcapsules having fragmented chromatin are subjected to RT reaction by suspending them in a suitable reaction mix (e.g., Maxima RT reaction mix from Thermo Fisher Scientific, cat no EP0753) containing the RT primer. Synthesize cDNA by incubating the microcapsules at a desirable temperature in the range of 4-80° C. and more preferably in the range of 37-50° C. and even more preferably at 42-50° C. After cDNA synthesis is complete, the transcriptome fraction becomes encoded in the cDNA molecules.

### Barcoding by Split-and-Pool:

Perform simultaneous barcoding of cDNA and tagmented DNA by ligating the barcoding DNA adapters following the principle described above (Performing rounds of split-pool on microcapsules carrying cDNA). After first round of split-pool, the microcapsules comprising the tagmented DNA and cDNA molecules are pooled and carried through additional rounds of barcode ligation. Perform a desirable number of split-and-pool rounds to attach barcodes to cDNA and tagmented DNA. In a preferred scenario no more than 8 rounds of split-pool reaction should be performed and more preferably in the range of 2-4 rounds of split-pool. Following the split-pool approach described here the genomic and transcriptomic fractions will share the same cell barcodes. Note that the nucleotide sequence at 3′ end of barcoded tagmented DNA, and 3′ end of barcoded cDNA are preferentially designed in such a way that they have different PCR adapter sequences. This feature enables selective enrichment for example after the first PCR amplification the PCR amplicons may be split into two portions for further enrichment and generation of genomic and transcriptomic libraries for sequencing.

### Adding PCR Adapters to Genomic and Transcriptomic Fractions:

After final round of split-pool reaction the genomic and transcriptomic fractions may be purified by washing the capsules in a suitable washing buffer. In some scenarios, at this step the nucleic acid fragments inside the microcapsules may be released into a bulk (e.g., by using protease enzyme) and purified using magnetic particles or purification columns. Next, the nuclei acid fragments may be subjected to gap filling and ligation reaction, during which DNA fragments encoding genomic fraction are tagged with Nextera adapter blocking oligo. Specifically, the use of Nextera adapter with 3InvdT modification at the 3′ end, blocks the template-switching reaction. As a result, the template-switching reaction may be performed only on a transcriptomic fraction using the TSO primer (5′-AAGCAGTGGTATCAACGCAGAGTGAATrGrGrG) (SEQ ID 8). Blocking of template switching reaction on genomic fraction may reduce the contamination of transcriptome libraries.

### Amplification of the Barcoded Genomic and Transcriptomic Fractions:

Wash the microcapsules comprising the barcoded genome and transcriptome fractions in a desirable buffer (e.g. PCR buffer). Transfer the solution to the PCR tube and initiate the PCR reaction. It may be preferable to use KAPA HiFi HotStart for performing the PCR reaction. Amplify the barcoded genome and transcriptome fractions by 8-14 rounds of PCR, preferably by 12 rounds of PCR and using universal PCR adapters. Wash the microcapsules to remove the unused primers from the first PCR. Noteworthy, amplification of the barcoded genome and transcriptome fractions may be also performed in bulk. Depending on the amount of primer-dimers and side products produced during the PCR one may choose to perform amplification of barcoded genome and transcriptome fractions in bulk, or in microcapsules.

### Separately Amplifying the Barcoded Genomic and Transcriptomic Fractions:

Treat the microcapsules with protease enzyme to release the barcoded nucleic acids to the bulk. Split the collected nucleic acids reaction into two even fractions. Purify the first fraction using ≥1.2× volumes SPRI beads. Purify the second fraction using ≤0.9× volumes SPRI beads. Perform the PCR on the first and second fractions separately using PCR indexing primers. The number of PCR cycles is 4-25 and preferably in the range of 9-12 cycles. At the end of this step one may have two DNA libraries, 1) a first library encoding the barcoded genomic (epigenomic) fraction, and 2) a second library encoding the barcoded transcriptomic fraction. These libraries may need additional round of purification and/or fragmentation depending on their quality and fragment size distribution.

### Fragment the Transcriptomic Library:

Perform fragmentation of the transcriptomic library. For fragmentation one can use enzymatic, physical or chemical means. In some scenarios it may be preferable to use enzymatic fragmentation (e.g., NEBNext Ultra DNA Library Prep Kit for Ilumina, cat no. #E7645) or a tagmentation reaction (e.g., Nextera XT DNA library preparation kit). After fragmentation the library is ligated to PCR adapter and amplified by PCR (preferably 9 to 12 rounds of PCR). The resulting library is purified using magnetic particles (e.g., SPRI beads).

### Sequencing:

Following the instruction of the DNA sequencing provider (e.g., Illumina) sequence the DNA libraries. When using Illumina sequencing instrument, the genomic fraction may be sequenced using Read 1—≥75 cycles, Read 2—≥90 cycles, Read 3—6-8 cycles, and Read 4—≥75 cycles. The transcriptomic fraction, may be sequenced using Read 1—≥70 cycles, Read 2—6-8 cycles, Read 3—≥100 cycles.

### Simultaneous Single-Cell Transcriptomics, Epigenomics and Genomics, Including Methylomics (Related to FIG. 5)

In one example, the method of the invention can be used to combine scRNA-Seq [35], scATAC-Seq [6], CUT&Tag [49] and bisulfite-free DNA methylation sequencing [50] methodologies to perform an integrated and high-throughput multi-omics analysis of single-cells. Critically, the cells have to be isolated to microcapsules before processing them through aforementioned methodologies. The compartmentalization of cells in microcapsules provides numerous analytical advantages, including enabling removal of intracellular inhibitors, allowing the performance of multi-step reaction on the same cell, and allowing the cells to be subjected to biochemical reaction conditions that are incompatible with existing methodologies.

As shown in FIG. 5, first the microcapsules comprising cells are generated as described above. Cells of interest are encapsulated into microcapsules and are lysed to release the nucleic acids. It may be desirable to use lysis conditions that are not detrimental, or only minimally detrimental, to chromatin structure. Upon cell lysis, the cells may be washed in a desirable buffer to remove lysis reagents and enzymatic reaction inhibitors. The microcapsules having nucleic acids from lysed cells may be dispersed in suitable reaction conditions to initiate the enzymatic reaction. The nucleic acids can be subjected to transposase-driven DNA fragmentation reaction (tagmentation). During tagmentation the open (accessible) chromatin regions are fragmented by transposase, which catalyzes insertion of the DNA adapters. These adapters can comprise nucleotide sequence that facilitates the capture of 1′ barcode during split-pool procedure. In addition, the specific epigenetic marks and regulatory proteins from the same cells can also be targeted, by Tn5 fused to target specific antibody. Upon binding the DNA sequence close to the target histone modification under antibody guidance, the Tn5 transposase fused to protein-A inserts the adapter. To distinguish double stranded DNA (dsDNA) ends generated during CUT&Tag approach Tn5 protein can be preloaded with unique indexed adapters. Using 11 unique indexes the said approach can be used to multiplex all 11 histone modifications that are known to exist in mammalian cells. After tagmentation of the genomic DNA (gDNA), the microcapsules are resuspended in another reaction mix to initiate cDNA synthesis from the mRNA. Once the open chromatin and DNA surrounding targets of interest (e.g. modified histones, transcription factors) are tagged with unique indexes the encapsulated cells are subjected to RT reaction. Performing these steps, the plurality of microcapsules will eventually comprise the material derived from a plurality of single cells and which corresponds to: i) transcriptome (in the form of cDNA), ii) open chromatin (in the form of indexed Tn5 tagmented DNA) and iii) DNA regulatory elements (in the form of indexed CUT&Tag tagmented DNA). In one example, the cDNA and gDNA fragments may be barcoded using a ligation-based combinatorial indexing strategy (as described in “Performing dsDNA barcoding by split-pool” and/or “Performing rounds of split-pool on microcapsules carrying cDNA” section) to simultaneously tag all nucleic acid fragments generated by the Tn5 transposases and the cDNA molecules generated during RT. The semi-permeable nature of shell of the microcapsules is critical in this multi-step process because it enables chemical and enzymatic treatment of nuclei acids, while retaining them inside. Treat the microcapsules with protease enzyme to release the barcoded nucleic acids to the bulk. Next, treat the nucleic acids with TET/pyridine borane for the detection of methylated cytosines, 5-mC and perform PCR. Amplify the nucleic acids using universal primers by 4-14 rounds of PCR, preferably by 12 rounds of PCR. Split the collected nucleic acids reaction into two even fractions. Purify the first fraction using ≥1.2× volumes SPRI beads. Purify the second fraction using ≤0.9× volumes SPRI beads. Perform the PCR on the first and second fractions separately using PCR indexing primers. The number of PCR cycles is 4-25 and preferably in the range of 9-12 cycles. At the end of this step one may have two DNA libraries, one encoding the barcoded genomic DNA corresponding to epigenomics and methylomics, and second encoding the barcoded transcriptome. The libraries may need additional rounds of purification and/or fragmentation. It may be desirable to perform additional fragmentation on the transcriptomic library as described above. After fragmentation the library is ligated to PCR adapter and amplified by PCR (preferably 9 to 12 rounds of PCR). The resulting library is purified using magnetic particles (e.g., SPRI beads). Following the instruction of the DNA sequencing provider (e.g., Illumina) the DNA libraries are sequenced. When using Illumina sequencing instrument, it may be preferable to sequence the barcoded genomics DNA fraction using Read 1—≥75 cycles, Read 2—≥90 cycles, Read 3—6-8 cycles, and Read 4—≥75 cycles. The barcoded transcriptomic fraction, may be sequenced using Read 1—≥70 cycles, Read 2—6-8 cycles, Read 3—≥100 cycles.

All publications, patents, and patent applications mentioned herein are hereby incorporated by reference in their entirety. In cases where the present specification and a document incorporated by reference include conflicting and/or inconsistent disclosure, the present specification shall control.

### REFERENCES

1. Klein, A. M., et al., Droplet barcoding for single-cell transcriptomics applied to embryonic stem cells. Cell, 2015. 161(5): p. 1187-201.
2. Macosko, E. Z., et al., Highly Parallel Genome-wide Expression Profiling of Individual Cells Using Nanoliter Droplets. Cell, 2015. 161(5): p. 1202-14.
3. Zheng, G. X., et al., Massively parallel digital transcriptional profiling of single cells.

Nat Commun, 2017. 8: p. 14049.
4. Satpathy, A. T., et al., Massively parallel single-cell chromatin landscapes of human immune cell development and intratumoral T cell exhaustion. Nat Biotechnol, 2019. 37(8): p. 925-936.
5. Buenrostro, J. D., et al., Transposition of native chromatin for fast and sensitive epigenomic profiling of open chromatin, DNA-binding proteins and nucleosome position. Nat Methods, 2013. 10(12): p. 1213-8.
6. Cusanovich, D. A., et al., Multiplex single-cell profiling of chromatin accessibility by combinatorial cellular indexing. Science, 2015. 348(6237): p. 910-4.
7. Lareau, C. A., et al., Droplet-based combinatorial indexing for massive-scale single-cell chromatin accessibility. Nat Biotechnol, 2019. 37(8): p. 916-924.
8. Domcke, S., et al., A human cell atlas of fetal chromatin accessibility. Science, 2020. 370(6518).
9. Lara-Astiaso, D., et al., Immunogenetics. Chromatin state dynamics during blood formation. Science, 2014. 345(6199): p. 943-9.
10. Mulqueen, R. M., et al., Highly scalable generation of DNA methylation profiles in single cells. Nat Biotechnol, 2018. 36(5): p. 428-431.
11. Luo, C., et al., Single-cell methylomes identify neuronal subtypes and regulatory elements in mammalian cortex. Science, 2017. 357(6351): p. 600-604.
12. Vitak, S. A., et al., Sequencing thousands of single-cell genomes with combinatorial indexing. Nat Methods, 2017. 14(3): p. 302-308.
13. Laks, E., et al., Clonal Decomposition and DNA Replication States Defined by Scaled Single-Cell Genome Sequencing. Cell, 2019. 179(5): p. 1207-1221 e22.
14. Peterson, V. M., et al., Multiplexed quantification of proteins and transcripts in single cells. Nat Biotechnol, 2017. 35(10): p. 936-939.
15. Stoeckius, M., et al., Simultaneous epitope and transcriptome measurement in single cells. Nature Methods, 2017. 14(9): p. 865-+.
16. Leonavicius, K., et al., Multi-omics at single-cell resolution: comparison of experimental and data fusion approaches. Curr Opin Biotechnol, 2019. 55: p. 159-166.
17. Granja, J. M., et al., Single-cell multiomic analysis identifies regulatory programs in mixed-phenotype acute leukemia. Nature Biotechnology, 2019. 37(12): p. 1458-+.
18. Zhu, C., et al., An ultra high-throughput method for single-cell joint analysis of open chromatin and transcriptome. Nat Struct Mol Biol, 2019. 26(11): p. 1063-1070.
19. Cao, J., et al., Joint profiling of chromatin accessibility and gene expression in thousands of single cells. Science, 2018. 361(6409): p. 1380-1385.
20. Furka, A., et al., General method for rapid synthesis of multicomponent peptide mixtures. Int J Pept Protein Res, 1991. 37(6): p. 487-93.
21. Maclean, D., et al., Encoded combinatorial chemistry: synthesis and screening of a library of highly functionalized pyrrolidines. Proc Natl Acad Sci USA, 1997. 94(7): p. 2805-10.
22. Rosenberg, A. B., et al., Single-cell profiling of the developing mouse brain and spinal cord with split-pool barcoding. Science, 2018. 360(6385): p. 176-182.
23. Cao, J., et al., The single-cell transcriptional landscape of mammalian organogenesis. Nature, 2019. 566(7745): p. 496-502.
24. Cao, J., et al., Comprehensive single-cell transcriptional profiling of a multicellular organism. Science, 2017. 357(6352): p. 661-667.
25. Cao, J., et al., Sci-fate characterizes the dynamics of gene expression in single cells. Nat Biotechnol, 2020. 38(8): p. 980-988.
26. Evers, D. L., et al., The effect of formaldehyde fixation on RNA: optimization of formaldehyde adduct removal. J Mol Diagn, 2011. 13(3): p. 282-8.
27. Lareau, C. A., et al., Massively parallel single-cell mitochondrial DNA genotyping and chromatin profiling. Nature Biotechnology, 2020.
28. O'Huallachain, M., et al., Ultra-high throughput single-cell analysis of proteins and RNAs by split-pool synthesis. Commun Biol, 2020. 3(1): p. 213.
29. Gierahn, T. M., et al., Seq-Well: portable, low-cost RNA sequencing of single cells at high throughput. Nat Methods, 2017. 14(4): p. 395-398.
30. Clark, S. J., et al., scNMT-seq enables joint profiling of chromatin accessibility DNA methylation and transcription in single cells. Nat Commun, 2018. 9(1): p. 781.
31. Dey, S. S., et al., Integrated genome and transcriptome sequencing of the same cell. Nat Biotechnol, 2015. 33(3): p. 285-289.
32. Macaulay, I. C., et al., G&T-seq: parallel sequencing of single-cell genomes and transcriptomes. Nat Methods, 2015. 12(6): p. 519-22.
33. Angermueller, C., et al., Parallel single-cell sequencing links transcriptional and epigenetic heterogeneity. Nat Methods, 2016. 13(3): p. 229-232.
34. Hu, Y., et al., Simultaneous profiling of transcriptome and DNA methylome from a 50 single cell. Genome Biol, 2016. 17: p. 88.
35. Hou, Y., et al., Single-cell triple omics sequencing reveals genetic, epigenetic, and transcriptomic heterogeneity in hepatocellular carcinomas. Cell Research, 2016. 26(3): p. 304-319.
36. Argelaguet, R., et al., Multi-omics profiling of mouse gastrulation at single-cell resolution. Nature, 2019. 576(7787): p. 487-+.
37. Zilionis, R., et al., Single-cell barcoding and sequencing using droplet microfluidics. Nat Protoc, 2017. 12(1): p. 44-73.
38. Marjanovic, N. D., et al., Emergence of a High-Plasticity Cell State during Lung Cancer Evolution. Cancer Cell, 2020. 38(2): p. 229-246 e13.
39. LaFave, L. M., et al., Epigenomic State Transitions Characterize Tumor Progression in Mouse Lung Adenocarcinoma. Cancer Cell, 2020. 38(2): p. 212.
40. Buenrostro, J. D., et al., Integrated Single-Cell Analysis Maps the Continuous Regulatory Landscape of Human Hematopoietic Differentiation. Cell, 2018. 173(6): p. 1535-1548 e16.
41. Ding, J., et al., Systematic comparison of single-cell and single-nucleus RNA-sequencing methods. Nat Biotechnol, 2020. 38(6): p. 737-746.
42. Habib, N., et al., Massively parallel single-nucleus RNA-seq with DroNc-seq. Nat Methods, 2017. 14(10): p. 955-958.
43. Amini, S., et al., Haplotype-resolved whole-genome sequencing by contiguity-preserving transposition and combinatorial indexing. Nat Genet, 2014. 46(12): p. 1343-9.
44. Ryan M. Mulqueen, B. A. D., Casey A. Thornton, Zeynep Sayar, Kristof A. Torkenczy, Andrew J. Fields, Kevin M. Wright, Xiaolin Nan, Ramesh Ramji, Frank J. Steemers, Brian J. and O. R. a. A. C. Adey, Improved single-cell ATAC-seq reveals chromatin dynamics of in vitro corticogenesis. https://www.biorxiv.org/content/10.1101/637256v1.full.pdf.
45. Zhu, C., S. Preissl, and B. Ren, Single-cell multimodal omics: the power of many. Nat Methods, 2020. 17(1): p. 11-14.
46. Rubin, A. J., et al., Coupled Single-Cell CRISPR Screening and Epigenomic Profiling Reveals Causal Gene Regulatory Networks. Cell, 2019. 176(1-2): p. 361-376 e17.
47. Mimitou, E. P., et al., Multiplexed detection of proteins, transcriptomes, clonotypes and CRISPR perturbations in single cells. Nat Methods, 2019. 16(5): p. 409-412.
48. Mimitou, E. P., Scalable, multimodal profiling of chromatin accessibility and protein levels in single cells. bioRxiv https://doi.org/10.1101/2020.09.08.286914, 2020.
49. Skene, P. J. and S. Henikoff, An efficient targeted nuclease strategy for high-resolution mapping of DNA binding sites. Elife, 2017. 6.
50. Skene, P. J., J. G. Henikoff, and S. Henikoff, Targeted in situ genome-wide profiling with high efficiency for low cell numbers. Nature Protocols, 2018. 13(5): p. 1006-1019.
51. Liu, Y. B., et al., Bisulfite-free direct detection of 5-methylcytosine and 5-hydroxymethylcytosine at base resolution. Nature Biotechnology, 2019. 37(4): p. 424.
52. Brangwynne, C. P., P. Tompa, and R. V. Pappu, Polymer physics of intracellular phase transitions. Nature Physics, 2015. 11(11): p. 899-904.

1.-53. (canceled)

54. A method for sequentially attaching a plurality of oligonucleotides to nucleic acids to produce barcoded nucleic acids, wherein the nucleic acids are obtained from a plurality of cells, wherein the plurality of cells are in a plurality of microcapsules, each microcapsule comprising a semi-permeable shell and a core, wherein each cell is in a separate microcapsule, the method comprising:
(a) lysing the cells within the microcapsules to release DNA and RNA inside the microcapsules;
(b) optionally in the microcapsule:
(i) converting released cellular RNA to cDNA by reverse transcription; and/or
(ii) fragmenting the released cellular DNA to produce fragmented DNA; and/or
(iii) amplifying fragmented DNA and/or cDNA,
(c) applying a combinatorial indexing strategy to attach the plurality of oligonucleotides one at a time to the released cellular DNA, the released cellular RNA, the cDNA, and/or the fragmented DNA and/or amplified DNA and/or cDNA in each microcapsule to produce barcoded nucleic acids;
wherein the barcoded nucleic acids produced in each microcapsule comprise a sequence of oligonucleotides that is specific to that microcapsule.

55. The method of claim 54, wherein the combinatorial indexing strategy of (c) comprises:
(i) randomly distributing the plurality of microcapsules into a plurality of compartments, wherein each compartment comprises more than one microcapsule;
(ii) processing the microcapsules in each compartment to attach an oligonucleotide to the nucleic acid in the microcapsules, wherein a different oligonucleotide is utilised in each compartment;
(iii) pooling and randomly re-distributing the plurality of microcapsules into a further plurality of compartments, wherein each further compartment comprises more than one microcapsule, and processing the microcapsules in each compartment to attach a further oligonucleotide to the nucleic acid in the microcapsules, wherein a different oligonucleotide is utilised in each of the further compartments; and
optionally repeating step (iii) one or more times.

56. The method of claim 54, comprising attaching a unique molecular identifier and/or an adapter to the released cellular RNA, the released cellular DNA or the barcoded nucleic acids, optionally wherein the adapter is a PCR adapter, and/or a sequencing adapter, and/or wherein the adapter comprises an endonuclease recognition sequence and/or a promoter sequence.

57. The method of claim 56, wherein the attaching comprises ligating or attaching via a nucleic acid extension reaction.

58. The method of claim 54, comprising breaking the plurality of microcapsules to release the barcoded nucleic acids under conditions that do not damage the barcoded nucleic acids.

59. The method of claim 56, comprising purifying and/or amplifying the released barcoded nucleic acids.

60. The method of claim 54, comprising producing a DNA library with the barcoded nucleic acids within the microcapsules or with the released barcoded nucleic acids.

61. The method of claim 59, comprising sequencing the barcoded nucleic acids.

62. The method according to claim 54, comprising labelling (hashing) the plurality of cells in the plurality of microcapsules prior to (a), preferably with DNA-antibody conjugates.

63. The method according to claim 54, wherein (b) comprises converting the released cellular RNA to cDNA by reverse transcription.

64. The method of claim 63, comprising ligating an oligonucleotide sequence to the released cellular RNA before reverse transcription.

65. The method according to claim 54, wherein (b) comprises fragmenting the released cellular DNA.

66. The method according to claim 65, comprising fragmenting the released cellular DNA by physical, chemical or enzymatic means.

67. The method of claim 66, comprising fragmenting the released cellular DNA using ultrasound or using complexes that generate hydroxyl radicals.

68. The method of claim 66, comprising fragmenting the released cellular DNA using enzymatic means, preferably a nuclease(s) and/or transposase and/or an enzyme fused to antibody.

69. The method of claim 54, wherein the semi-permeable shell allows diffusion of a reagent, an enzyme and/or a substrate for the method steps through the shell, while retaining the nucleic acids, optionally wherein the substrate is a barcoding oligonucleotide, or a PCR primer.

70. The method of claim 54, wherein the semi-permeable shell allows for diffusion of smaller molecular weight compounds of approximately MW 200,000 or less through the shell, while retaining larger molecular weight compounds of approximately MW 300,000 and above.

71. The method of claim 54, wherein the semi-permeable shell is permeable to compounds having a molecular weight of 120±80 kDa or lower.

72. The method of claim 54, wherein the semi-permeable shell comprises a gel formed from a polymer, wherein the polymer in the gel is covalently cross-linked.

73. The method of claim 72, wherein the polymer is a polyampholyte and/or a polyelectrolyte or a synthetic polymer.
